(19) United States
(12) Patent Application Publication
(10) Pub. No.: US 2023/0270389 A1
(43) Pub. Date: Aug. 31, 2023

(54) TELEMEDICINE SYSTEM

(71) Applicant: MEDAICA INC., Las Vegas, NV (US)

(72) Inventors: Stephen RANDALL, Las Vegas, NV (US); Mark GRETTON, Las Vegas, NV (US); Dan GIESCHEN, Las Vegas, NV (US); Nick GIESCHEN, Las Vegas, NV (US); Pablo RIVAS, Las Vegas, NV (US); Augusto GARCIA, Las Vegas, NV (US)

(21) Appl. No.: 18/024,142

(22) PCT Filed: Aug. 31, 2021
(86) PCT No.: PCT/US2021/048415

§ 371 Date: Mar. 1, 2023

Related U.S. Application Data
(60) Provisional application No. 63/073,207, filed on Sep. 1, 2020.
(60) Provisional application No. 63/110,446, filed on Nov. 6, 2020.
(60) Provisional application No. 63/147,428, filed on Feb. 9, 2021.

(51) Int. Cl. A61B 5/00; A61B 7/04; G10L 25/84; H04R 5/027; H04R 3/00; H04S 1/00; G10L 21/0216; H04R 1/46

(52) U.S. Cl. CPC A61B 5/7465; A61B 5/0022; A61B 7/04; A61B 5/7203; G10L 25/84; H04R 5/027; H04R 3/005; H04S 1/007; G10L 21/0216; H04R 1/46; A61B 2562/0204; H04S 2400/15; H04S 2400/13; G10L 2021/02165

(57) ABSTRACT

A telemedicine system comprises (i) medical devices that are each configured to generate patient datasets, and (ii) a remote web server. At least one of the medical devices is then configured to upload or send patient datasets to the remote web server, directly from an internet-connected app running either on the medical device or on at least one intermediary device. The remote web server is configured to generate a unique web-link that is associated with a specific patient dataset. The unique web-link enables a healthcare professional to review the specific patient dataset by selecting the web-link from within a web browser or from within any dedicated telemedicine application that opens web-links.

### CROSS-REFERENCE TO RELATED APPLICATIONS

This application claims priority to U.S. Provisional Application No. 63/073,207, filed Sep. 1, 2020; U.S. Provisional Application No. 63/110,446, filed Nov. 6, 2020; and U.S. Provisional Application No. 63/147,428, filed Feb. 9, 2021, the entire contents of each of which being fully incorporated herein by reference.

### BACKGROUND OF THE INVENTION

### 1. Field of the Invention

The field of the invention relates to a telemedicine system including multiple medical devices and a remote server. Telemedicine systems enable remote diagnostics and clinical caring for patients, i.e. when a health professional and patient are not physically present with each other. Telehealth is generally thought of as broader in scope and includes non-clinical health care services; in this specification, the terms ‘telemedicine’ and ‘telehealth’ are used interchangeably and so ‘telemedicine’ should be broadly construed to include telehealth and hence include remote healthcare services that are both clinical and non-clinical.

A portion of the disclosure of this patent document contains material, which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### 2. Description of the Prior Art

Many appreciate that telemedicine is more than just using Skype®, Zoom®, or Facetime®, so that a doctor can look a Patient in the eyes. For telemedicine to be truly useful, the Patient must be able to collect and transmit a variety of data the healthcare professional needs to assess the Patient's health.

Although telemedicine can easily leverage patient-collectable data from simple and affordable devices, such as blood pressure cuffs, heart monitors, pulse oximeters and thermometers, etc., current solutions fail to provide uniform or easy ways for healthcare professionals to acquire more subjective or useful information from patients without a doctor's or nurse's supervision e.g. listening to a patient's body sounds (auscultation), taking an EKG or performing an ultrasound. Consequently, the inability for telemedicine platforms to easily interoperate with web-enabled electronic/digital medical devices (“Digital Medical Devices” or “DMDs”, also called simply “medical devices’ in this specification) has been an inhibitor of telemedicine advancing beyond the use of simple diagnostic sessions, mental health and dermatology.

In an ever-connected world, with increased fears of infections being spread in doctors' waiting rooms and hospitals, especially in light of the COVID-19 pandemic, patients and healthcare professionals alike need easier, more secure and/or interoperable telemedicine solutions.

Current telemedicine devices are not patient centric. They have been designed for healthcare professionals and few have been cleared by the FDA to be sold to consumers. In addition, the end-to-end experience often competes with and/or is too complex to use with existing telemedicine systems.

As demand for telemedicine increases, not least due to COVID-19, there is a drive to reduce the cost of devices as well as improve the quality and utility of services. The opportunity to truly democratize telemedicine will be unlocked when medical devices are much more affordable, and easy to use with any telemedicine platform.

### SUMMARY OF THE INVENTION

The invention, in a first aspect, is a telemedicine system comprising multiple medical devices, such as digital stethoscopes, digital blood pressure monitors and other medical and digital medical devices. An individual patient might use one or more of these devices in a telemedicine session with a healthcare professional. But the system is highly scalable and could include thousands, or tens of thousands of these devices, distributed across a population. The medical devices are each configured to generate patient datasets and are each configured to upload or send patient datasets to one or more remote web servers, directly from an internet-connected app running either on the device itself or on an intermediary device. The remote web server is configured to generate a unique web-link that is associated with a specific patient dataset. The unique web-link enables a healthcare professional to review the specific patient dataset by selecting the web-link from within a web browser or from within any dedicated telemedicine application that opens web-links. Additionally, or alternatively, the unique web-link enables a healthcare professional to initiate a virtual examination of the patient by selecting the web-link, which then leads to the opening of a link to a virtual examination room hosted on the remote web server.

We can generalise to:

A telemedicine system comprising one or more medical devices that are each configured to generate patient datasets, and a remote web server; in which:
a medical device is configured to upload or send patient datasets to the remote web server, directly from an internet-connected app running either on the medical device or on an intermediary device;
the remote web server is configured to generate a unique web-link that is associated with a specific patient dataset;
and in which the unique web-link enables a healthcare professional to review the specific patient dataset by selecting the web-link from within a web browser or from within any dedicated telemedicine application that opens web-links.

A second aspect is a telemedicine system comprising: multiple medical devices that are each configured to generate patient datasets, and a remote web server connected to each medical device; in which:
a medical device is configured to upload or send patient datasets to the remote web server, directly from an internet-connected app running either on the device or on an intermediary device;
and in which the medical device includes (i) a speech microphone configured to detect and/or record patient speech and (ii) a second microphone in the medical device configured to detect and/or record clinically relevant sounds and generate an audio dataset from those sounds;
and in which the internet-connected app is configured to treat that patient speech separately from the audio dataset and is hence configured to enable real-time voice communication from the patient to the healthcare professional at the same time as the audio dataset is being shared with the healthcare professional via the remote web server;
and the system is configured to enable the healthcare professional to select whether to listen to real-time voice communication from the patient or to listen to the audio dataset in real-time by muting, fully or partly, either the real-time voice communication or the audio dataset.

A third aspect is a medical device that includes (i) a speech microphone configured to detect and/or record patient speech and (ii) a second microphone configured to detect and/or record clinically relevant sounds and generate an audio dataset from those sounds;
in which the speech microphone uses one channel of a stereo channel pair, and the second microphone uses the other channel, and each channel is processed substantially in parallel or simultaneously.

A fourth aspect is a telemedicine system comprising multiple medical devices that are each configured to generate patient datasets, and a remote web server; in which:
one or more medical devices are each configured to upload or send patient datasets to the remote web server, directly from an internet-connected app running either on the device or on an intermediary device;
and in which the remote web server hosts or enables access to an applet that, when run on a patient's internet-connected app, provides instructions or guides to the patient to perform specific healthcare management protocols.

A fifth aspect is a telemedicine system comprising multiple medical devices that are each configured to generate patient datasets, and a remote web server connected to each medical device; in which:
at least one medical device is configured to upload or send patient datasets to the remote web server, directly from an internet-connected app running either on the device or on an intermediary device;
in which the system is configured to enable a healthcare professional and a patient to communicate via a virtual examination room, and the system is further configured to display a user interface that includes a virtual or graphical body image or body outline and one or more target positions at which a medical device is to be positioned by the patient;
and the system is further configured to enable a dynamic interaction between the patient or the healthcare professional and the user interface, to enable the patient to correctly position the medical device at the target position or positions.

The invention is implemented in a system called the Medaica system, which is described in the following sections.

### BRIEF DESCRIPTION OF THE FIGURES

Aspects of the invention will now be described, by way of example(s), with reference to the following Figures, which each show features of the invention:

FIG. 1 is a simplified cross section of a digital electronic stethoscope.

FIG. 2 is a simplified top view and cross section of a digital electronic stethoscope.

FIG. 3 is a simplified diagram of the electrical design of the electronic stethoscope

FIG. 4 is a diagram of some of the key players interacting with the Medaica system.

FIG. 5 is a diagram of the Medaica platform.

FIG. 6 is a system overview of one implementation of the invention.

FIG. 7 is a diagram illustrating a patient's journey.

FIG. 8 is a diagram illustrating a patient's journey.

FIG. 9 is a diagram illustrating a doctor's journey.

FIG. 10 is a diagram illustrating a user's interaction with the playback page.

FIG. 11 shows an example of a patient's web-app displaying an outline of a torso along with a video feed.

FIG. 12 shows another example of a patient's web-app with the graphical interface of a self-exam heart mode.

FIG. 13 shows a patient's web app displaying a countdown and recording quality window.

FIG. 14 shows a patient's web app displaying the torso outline shows when each auscultation position is recorded successfully

FIG. 15 is a flow diagram summarizing the steps of the self-exam procedure.

FIG. 16 shows a patient's web app displaying a specific exam procedure overlaid over a live video image of the user.

FIG. 17 shows a graphical interface of front lungs self-examination including a torso outline of a front torso and the required examination positions.

FIG. 18 shows a graphical interface of back lungs assisted examination including a torso outline of a back torso and required examination positions.

FIG. 19 shows a graphical interface of a video-positioning mode.

FIG. 20 shows a simplified flow diagram illustrating when an exam starts.

FIG. 21 shows a flow diagram illustrating the different steps according to a self-examination mode, custom examination mode or guided examination mode.

FIG. 22 shows a diagram illustrating the system key components.

FIG. 23 shows photographs illustrating several digital stethoscope devices.

FIG. 24 shows photographs illustrating a number of digital stethoscope devices.

FIG. 25 shows photographs illustrating a digital stethoscope device including a dummy socket (210).

FIG. 26 shows top-down, side and bottom up views (respectively, descending) of a digital stethoscope device.

### DETAILED DESCRIPTION

In one implementation of the invention, systems and methods are provided to enable a healthcare professional to conduct a remote exam from any web-enabled audio and/or video platform, not only simplifying telemedicine consultations that would otherwise require special devices and/or integration of disparate systems but also increasing the value of the telemedicine consultation. The systems and methods produce unique links that are exchanged between patients and healthcare professionals to either review files, such as but not limited to a patient's auscultation sounds, or for the patient to participate in a virtual exam. The unique link can also be used to control access rights, privacy and enable additional services, such as but not limited to diagnostic analysis, research and verification. The links can additionally contain rules, such as but not limited to permitting third party access right, sharing/viewing rules and financial controls such as but not limited to subscription usage and per user limits.

We will now describe examples of problems or challenges that have been addressed by implementations of the present invention, such as interoperability and security and tracking usage/permissions.

### Interoperability

Interoperability is an often overlooked but major challenge for telemedicine systems. Although the FDA recognizes the challenges of medical device interoperability (see fda.gov/medical-devices/digital-health/medical-device-interoperability) and mentions that devices with the ability to share information across systems and platforms can improve patient care, reduce errors and adverse events and encourage innovation, the problems of interconnectivity between digital medical devices (DMDs) and telemedicine systems can be far more subtle yet complex.

Telemedicine platforms do not provide uniform or easy support for multiple DMDs. Likewise, many DMDs will not work with any telemedicine systems without extensive (and often expensive) technology integration work. This is clearly a problem for both sides of the healthcare value-chain; healthcare professionals would ideally like telemedicine to support the use of most if not all the tools they use in their typical patient exams. If a telemedicine system doesn't support all their tools, its utility is limited.

These and other related problems are currently inadequately addressed by:
1) DMD manufacturers supplying their own closed/proprietary telemedicine solutions. However, such approaches are not very scalable as every doctor or hospital wishing to use that DMD will be unable to do so with their existing telemedicine solution or they will be forced to have multiple telemedicine solutions for every DMD. Furthermore, such solutions compete with telemedicine systems, so are unlikely to be widely embraced by those platforms.
2) Integrating the DMD into each telemedicine platform via the telemedicine platform's Application Program Interfaces (APIs). The problem with this approach is that with many hundreds of telemedicine solutions and thousands of specific implementations/configurations, each DMD manufacturer would have a very difficult and expensive task to integrate and maintain the end-to-end experience, having to potentially test and update software and hardware every time each telemedicine system is updated.

In addition, as DMDs leverage mobile technology and use wireless interfaces such as Bluetooth, primarily designed for consumers, they fail to address usability problems for healthcare professionals including a) a doctor might not wish to use a private device (their own phone) while examining a patient—that phone might ring with a personal call, and it is not ideal for sharing if they only have one DMD in the clinic and b) Bluetooth can be difficult to use when there is other radio-enabled equipment near-by or metal objects. Furthermore, if a user is talking with a doctor over any web-enabled video channel, and they then turn on a Bluetooth DMD, the most likely scenario is that the DMD will take over the audio channel resulting in the patient being unable to talk to or hear the doctor (the audio will be routed to the DMD). This is a solvable solution, but requires an interface that can negotiate between the telemedicine and DMD audio channels and switch between them manually or automatically, but in a way that does not confuse the patient or doctor. In a time-sensitive consultation, neither the doctor nor the patient want to waste time with complex interfaces and will undoubtedly be put off the experience if that happened.

Devices that have either not considered the above scenarios and related user experience issues from the start of their design, are invariably ill-suited to telemedicine.

### Security and Tracking Usage/Permissions

With each conventional DMD typically being a closed/proprietary system and with HIPAA (The Health Insurance Portability and Accountability Act of 1996) and GDPR (The General Data Protection Regulation 2016/679) requirements, there is a very complicated and politically charged problem to solve. The Medaica solutions provide an intermediary web-hub that operates separately from the telemedicine platform, and can, in its simplest form, work on any web-enabled system and can be simply accessed by a doctor and/or patient as a new window alongside their existing chosen telemedicine or video/chat/messaging solution, without requiring further integration. This is further enabled with secure web-enabled links that can grant access rights to connect permitted parties and provide features to securely share, review, authenticate files, export files and set rules over timing, sharing rights and business models, payments etc.

We will now describe the Medaica M1 DMD. M1 is a low-cost digital stethoscope that is aimed at telemedicine applications, rather than as a replacement for traditional stethoscopes. As such, it is aimed at the patient rather than the healthcare professional. A more detailed description of M1 now follows.

### M1 Low-Cost Digital Stethoscope

Medaica's system is designed to be hardware agnostic, however, today, there is no plug and play device that will result in the simple functionality and affordability required. To that end, Medaica is producing a simple electronic stethoscope, the M1. A target retail price is for example under $50. A target material cost (bill of materials) is for example under USD $15.

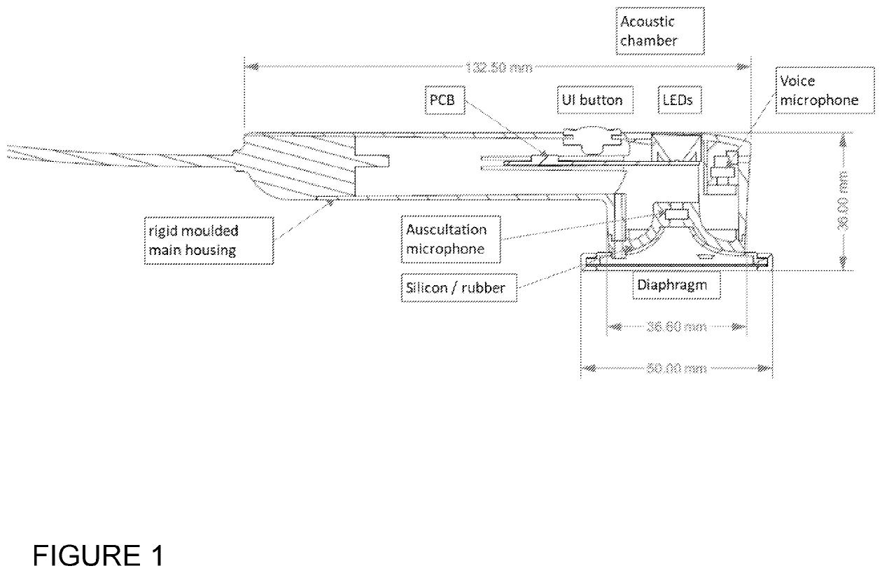

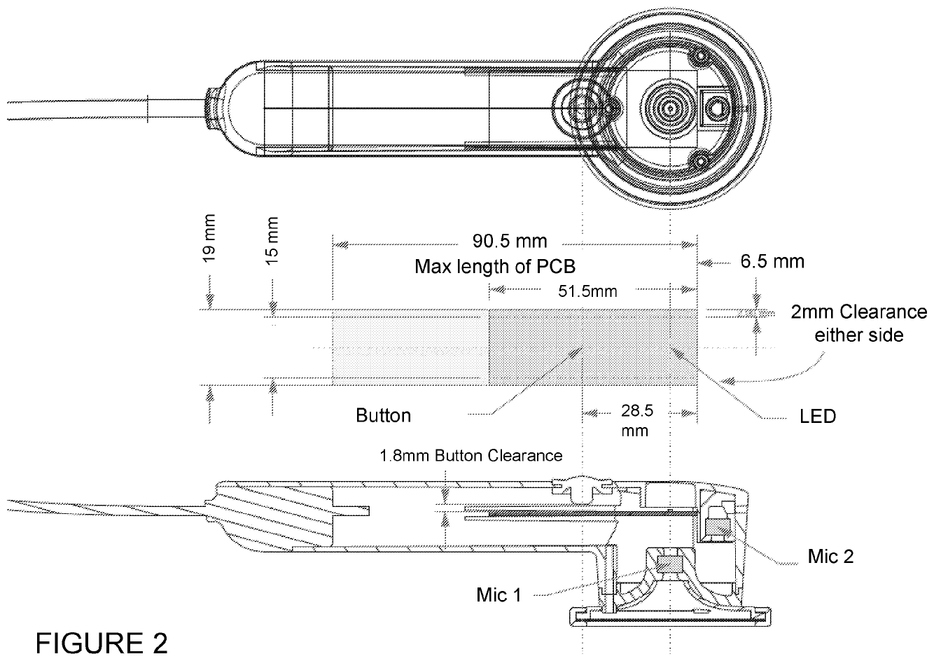

FIG. 1 shows a simplified cross section of M1 including examples of dimensions. FIG. 2 shows a top view and another cross section of the device including further examples of dimensions. M1 includes a USB microphone. It is mounted in a rigid molded enclosure. The enclosure is in the basic shape of a stethoscope. The front face has a traditional stethoscope diaphragm sealed onto an acoustic chamber into which a microphone, such as an electret or piezo microphone is mounted. In addition to the stethoscope microphone, a second microphone for patient voice, for detecting whether background noises are too loud and could affect the stethoscope microphone, and for noise cancelling, is mounted facing upwards towards the user.

These two microphones are connected respectively to the left and right channels of the USB stereo microphone channel so they can be processed in parallel.

On the rear face, a small “I'm alive” white LED, a “now recording” red LED, and a single user push button are mounted. The device is washable, so the LEDs and button are waterproof (IPX6) and fabricated as a simple membrane, like many medical and household cookery products. The various electrical items are connected to a USB audio bridge IC mounted on a small PCB. The device is large enough to be comfortable in the hand and therefore may contain a significant amount of empty space. This could be filled with ballast to improve the weight and feel of the device. Alternatively, the space may be used for more electronics components and a rechargeable lithium cell battery in more sophisticated versions. Furthermore, the design leaves the head of the device easily viewable when held by the patient, such that in a telemedicine consultation the patient will be able to be guided, either by the user interface or the healthcare professional, to move the head of device over specific auscultation target areas.

### M1 Connectivity

The initial design for M1 is a USB 2 wired design. Additionally, the device may also support Bluetooth (BT) connectivity. Adding BT connectivity would enable connectivity to supported device platforms and would add the following components: BT transceiver, ISM band antenna, microcontroller capable of implementing BT stack and application level encryption, power management device and battery plus some more UI elements and potentially an MFI chip. With USB 2 connectivity only, M1 is compatible with a number of platform or devices, such as: Windows laptops and PCs, Apple laptops and PCs, Android tablets and some phones (with a USB 2 to USB C adapter which is readily available) and Apple phones with a Lightning to USB converter and MFI device.

### M1 Mechanical Design

The main housing is formed from a target maximum of two injection molded plastic parts. These parts are molded from high density medical grade plastic and have sufficiently thick wall sections as to be acoustically stable. These plastic parts may be finished or plated to give a comfortable and durable finish.

### M1 Electrical Design

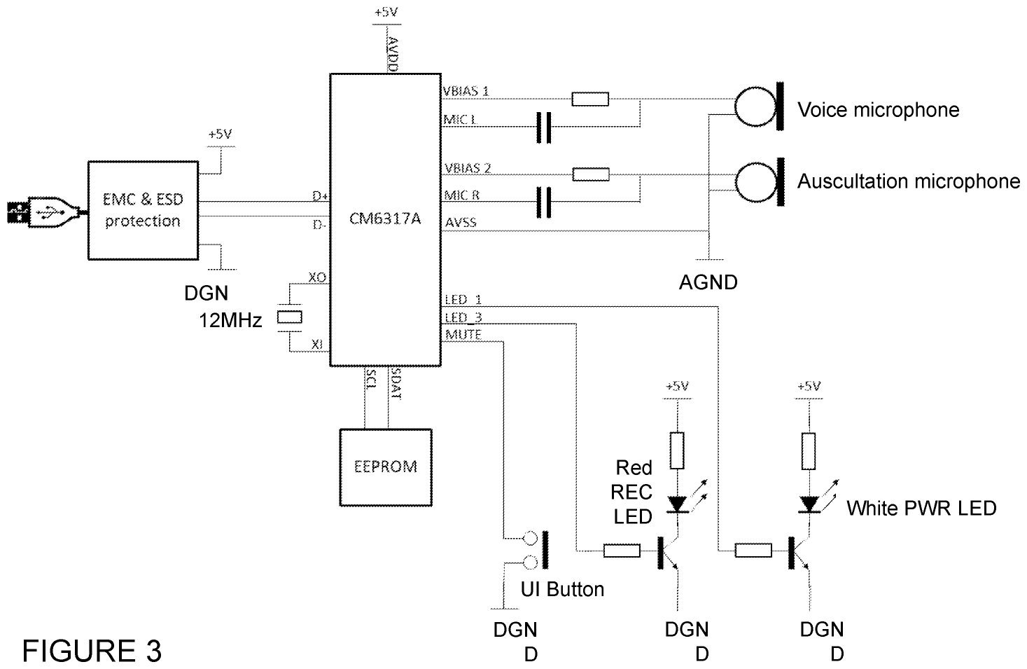

The electronic design is based around a standard USB to audio bridge IC from (e.g. CMedia CM 6317A). The Left and Right channels are used for the voice and auscultation microphones respectively. FIG. 3 shows a simplified diagram of the electrical design.

### M1 UX Philosophy

Like the hardware, the software must be simple. The website and mobile app can be used by users in “Guest” mode without any user login or sign up. This minimizes additional UX steps which could be life-saving if the user has an emergency and wants the fastest route to getting advice. The website and/or mobile app recognizes that the M1 device is plugged in (and will indicate if it is not) and can then guide the user on next steps.

### M1 Users

Users of medaica.com include, but not limited to:
Patients at home, such as consumers who directly connect M1 to PC, Mac or iOS or Android platforms to record heart and/or lung sounds.
Healthcare practitioners working remotely from patients. They have access to M1 files and can listen to them asynchronously or live on any web-enabled platform.
Researchers/analysts/specialists, subject to access rights to M1 files in order to diagnose, tag sounds, conduct research, teach, and/or to help ML/AI systems learn.
Hospitals/doctors requiring hosted solutions, such as healthcare practitioners who desire access rights to M1 files within their own networks and security requirements.
Assisted living/nursing homes where doctors may visit on a periodic basis, but can still be informed via forwarded auscultation data.

### Platform Overview

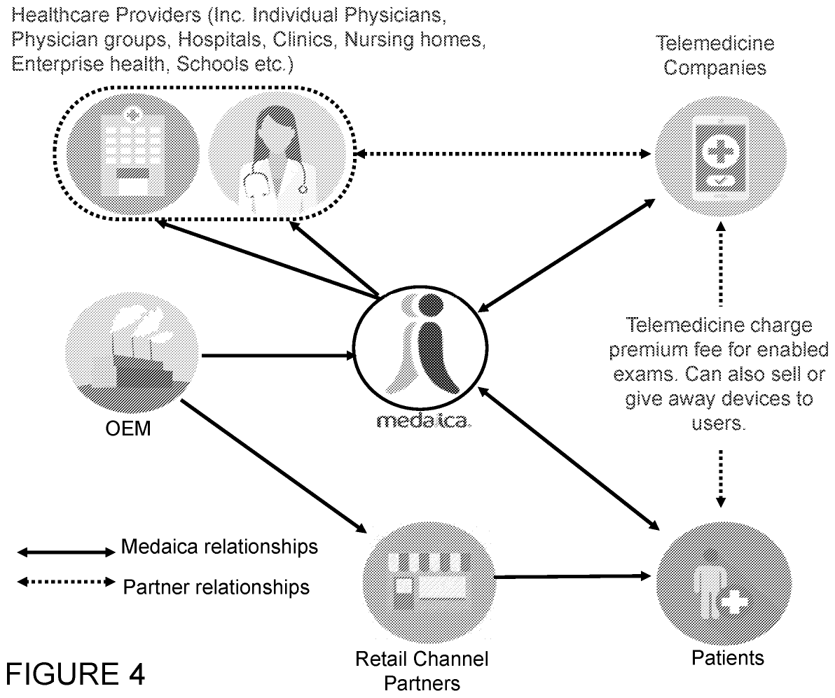

FIG. 4 shows a diagram illustrating the different players interacting with the Medaica system.

The Medaica system offers a number of product differentiation features, including but not limited to:

For Healthcare professionals:
Interoperability: Plug and Play solution, works with any existing telehealth system without the need to change systems, workflow, processes or procedures.
Adds value to telehealth exams by adding more capabilities (extending the clinical exam to the patient's home).
No Diagnostics: focus is on simplicity and on end-to-end utility, not on tech or artificial intelligence (AI).
Alternatively, diagnosis analysis, including AI diagnosis may be provided as an additional service.

For Telehealth platforms and other healthcare service providers/developers/device companies:
Extend platform utility with new services offerings.
Rapid integration and an optional integration via APIs.
Increase value to all users.
Data=value=improved/stickier services and capabilities.
Enable incremental revenue (by extending service and/or reach capabilities).
Increase clinical data, insights.
Roadmap for 3rd party hardware devices, AI services and EMR providers/integrators.

For Patients
A simple device: e.g. No Bluetooth™ to pair, no battery to charge.
Virtual clinic (on demand exam services) added to existing Telehealth experience.
Works with any video/messaging platform and device (e.g. Zoom™, Facetime™ Teladoc™, on a PC, MAC™, iPhone™, Android™).
No medical knowledge needed to operate device or software.
No subscription fees (business model is for exams to be charged via telehealth/data services).
Designed for consumer use.
As safe and easy to use as other consumer medical devices—e.g. blood pressure devices and PO2 devices.
Minimal information required from user (e.g. can use system as guest).

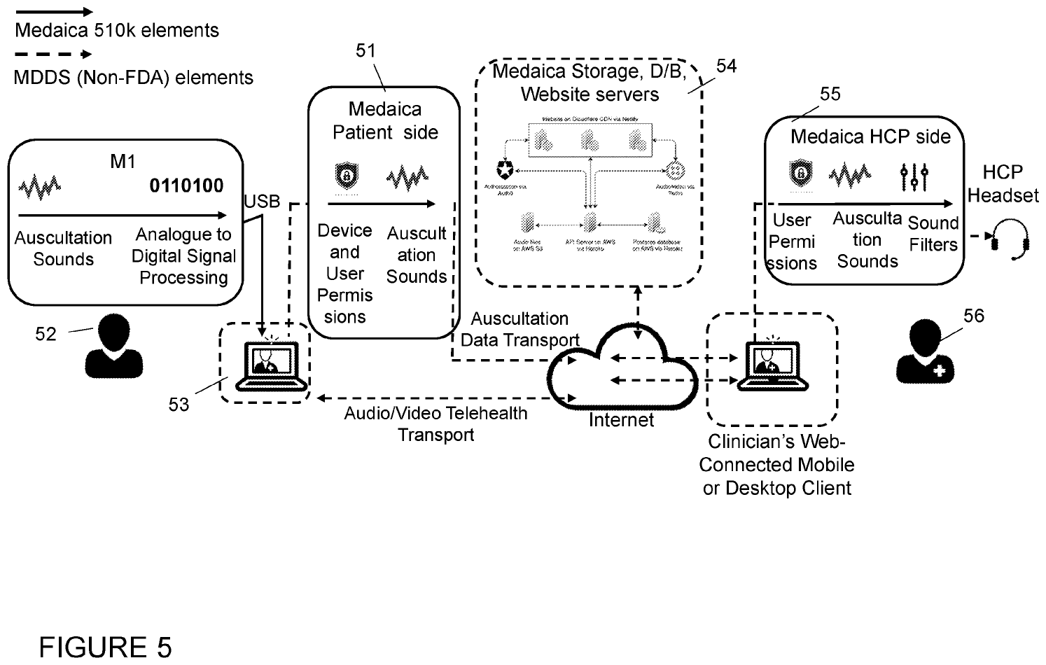

FIG. 5 shows a diagram of the system's platform. At the patient side (51), a patient (52) connects a Medaica M1 stethoscope to a USB port of the patient's Web-connected mobile or desktop client (53). The patient enters the Medaica Patient Side (51). The software recognizes Medaica M1 UDID and enables recording of auscultation sounds.
In Live mode, a health care professional (HCP) generates and sends an exam room passcode to the patient. Once the patient enters the passcode, the HCP can direct the patient and initiate recording.
In Store and Forward mode, the patient records auscultation sounds, guided by UI and can then send a unique link to those sounds to the Healthcare Professional (HCP).

Auscultation sounds are transmitted via Medaica Servers (54). The auscultation sounds web-link is sent to the HCP side.

At the HCP side (55), the HCP (56) visits the Medaica HCP Side.
In Live mode, the HCP generates and sends an exam room passcode to the patient. Once the patient enters the passcode, the HCP can direct the patient and initiate recording.
In Store and Forward mode, a link to the patient's sounds is sent to the HCP for review.

The HCP can choose to listen to auscultation sounds filtered or unfiltered and share, comment and/or export sounds, according to permissions.

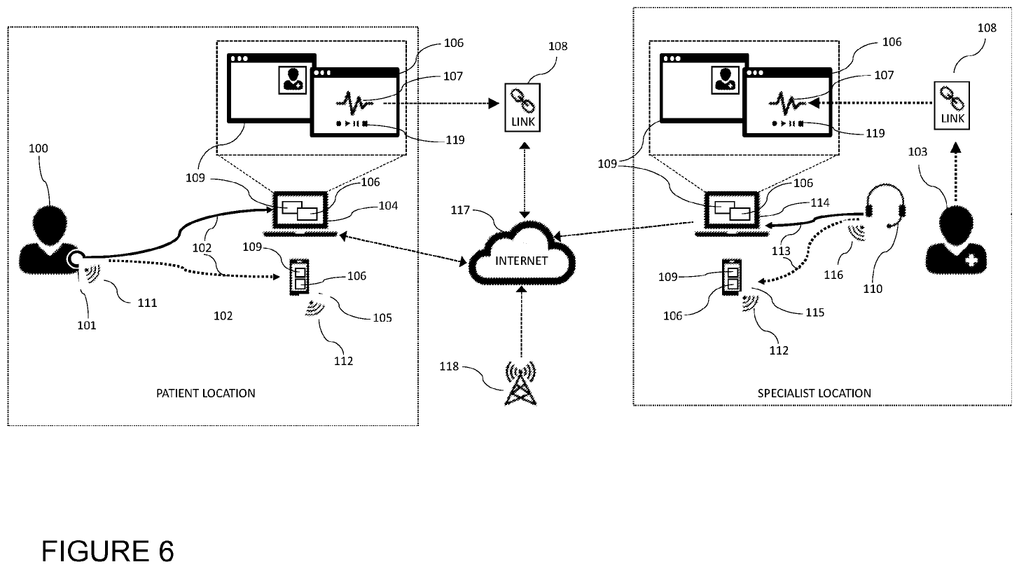

FIG. 6 illustrates a further example of the interactions within the Medaica system. A patient (100) is located at a remote location from the health care professional HCP (103).
101 is a web-enabled electronic medical device used for auscultation of body sounds.
102 is a cable connecting the electronic medical device (101) to either a web-enabled computing platform (104) or mobile phone (105).
103 is a healthcare professional such as but not limited to a doctor (and interchangeably referred to as a specialist and/or clinician in this document) at a different location than the patient.
104 is a web-enabled computing platform such as but not limited to a laptop.
105 is a mobile phone (or other such mobile computing platform), connected to the Internet via cellular or other wireless interconnectivity such as WiFi.
106 is a website (in this embodiment, medaica.com) for recording, storing and controlling access to patients' uploaded files, such as but not limited to auscultation files. This website can be viewed on any web-enabled devices such as the patient's laptop (104) or mobile phone (105) or the healthcare professional's laptop (114) or mobile phone (115).
107 is an example sound file recording via a patient's web-enabled electronic medical device.
108 is a web-enabled link controlling access to a patient's auscultation files.
109 is a web-enabled video or telemedicine site. This web-enabled site can be viewed on any web-enabled devices such as the patient's laptop (104) or mobile phone (105) or the healthcare professional's laptop (114) or mobile phone (115).
110 is a headset and mic set enabling better listening/talking experience for the healthcare professional.
111 is wireless connectivity for the electronic medical device, such as but not limited to Bluetooth or WiFi.
112 is cellular connectivity to/from the mobile phone to the cellular network (118).
113 is a cable connecting the headset and mic (110) to either the doctor's web-enabled computing platform (114) or mobile phone (115).
114 is a web-enabled computing platform such as but not limited to a laptop at the doctor's location.
115 is a mobile phone connected to the Internet via cellular or other wireless interconnectivity, such as WiFi at the Doctor's location.
116 is wireless connectivity for the healthcare professional's headset and mic 110
117 is the internet. 118 is a cellular network, connected to the internet (117).
119 is a record/play pause/stop example for recording and reviewing a sound file (107).

Examples of user journeys are now described.

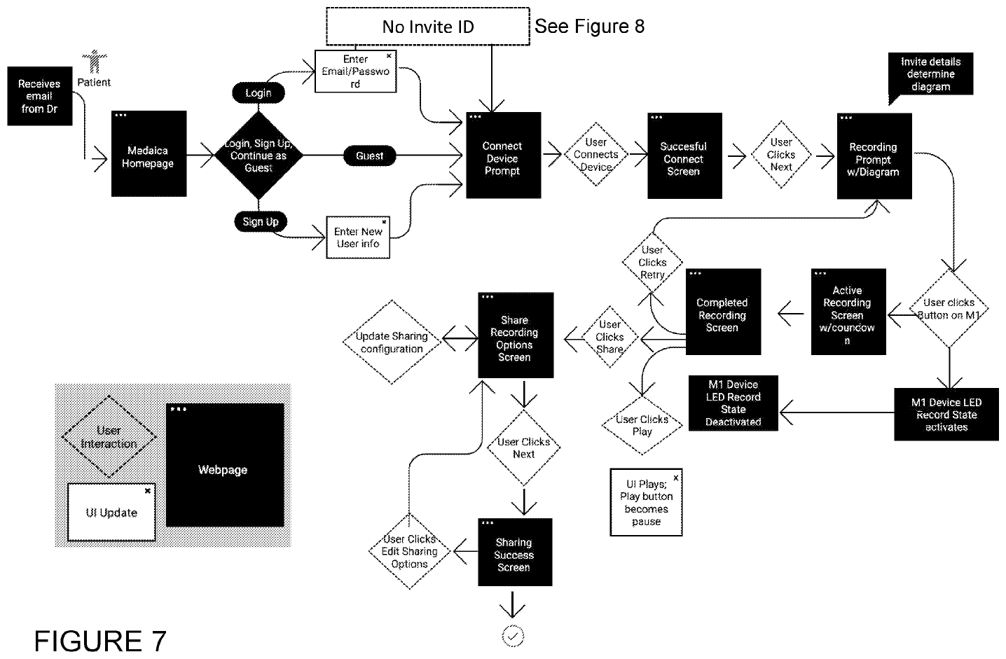

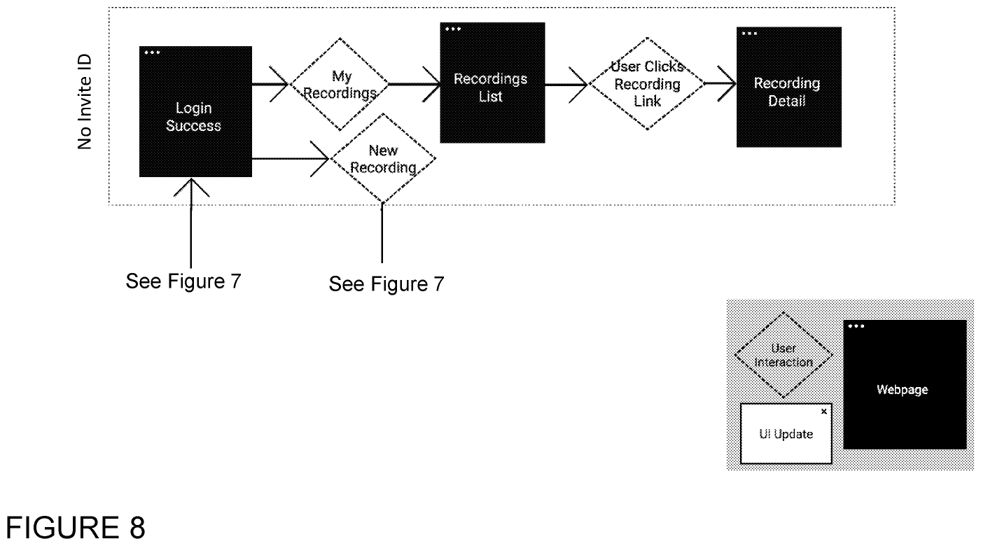

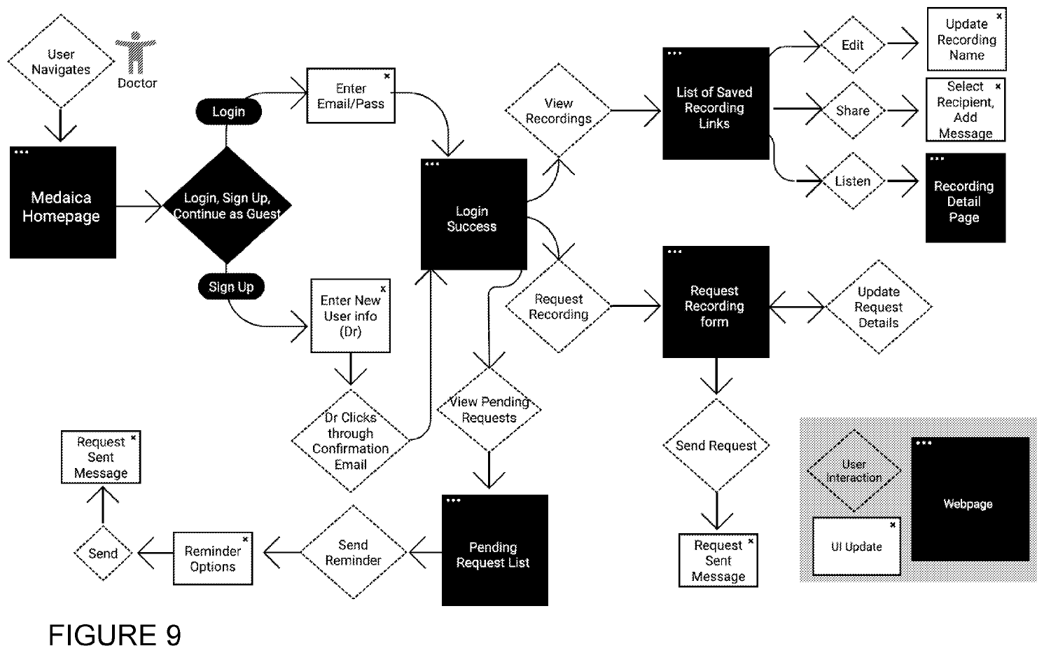

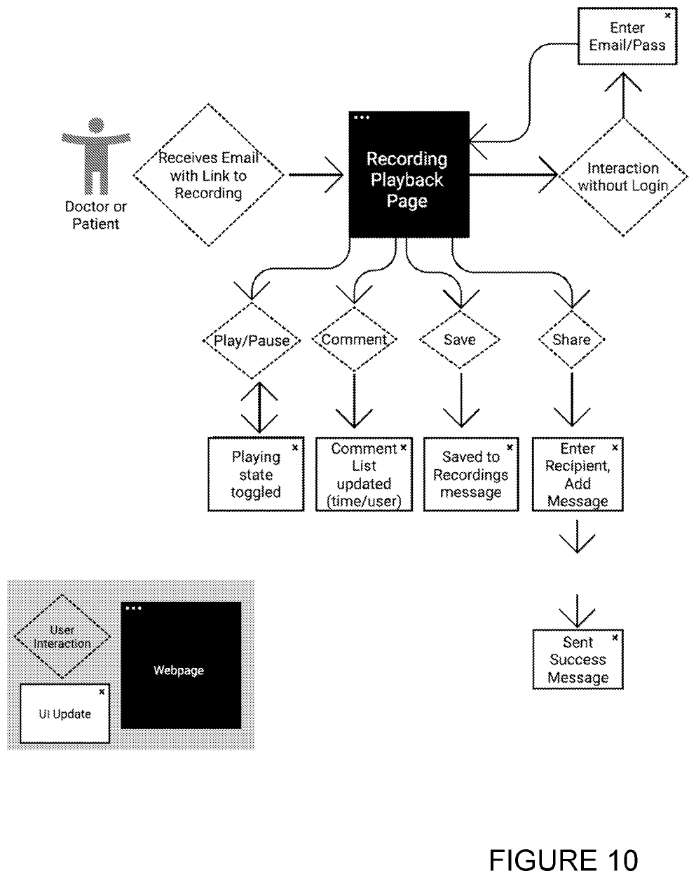

### Use Case 1—Store and Forward (See FIGS. 6 to 10)

As shown in FIG. 6, the Medaica website (106) displays simple instructions for the user (100) to connect and record auscultation sounds from the M1 device (101).
1. “Plug M1 into the USB port of your <PC, Mac, iPhone or Android>”.

When the M1 device is plugged into the USB port of the web-enabled PC or mobile device (104 or 105), the M1 LED is on constantly, medaica.com recognizes it and displays an icon showing it is plugged in and guides the user to the next steps. (If the M1 device is plugged in already, then #1 doesn't display).

Alternatively, the device (101) may be wirelessly connected, using for example Bluetooth, to the web-enabled PC or mobile device, which consequently would provide additional steps in the user journey.
2. “Using the Exam Positions diagram (not shown), place M1 on a position, then press the Record Button on M1.”

Alternatively, a start/stop record button (119) is provided on the website.

Alternatively, the user is guided via an Augmented Realty (AR) application, into position.

The M1 device is recognized by the web-enabled platform's camera (either directly via its shape, color etc., or via an identifying mark/code on M1). Once recognized by the system, the system shows the user when M1 is over a position to collect sounds, and either auto-start recording (optionally first showing a countdown) or highlight a start/stop recoding button.

The User places the M1 device on a position and presses the M1 record button. M1 LED displays red flashing.

A timer on the website UX displays a countdown (say 20 secs) (This could be greyed out if the M1 device is not plugged in to help the user understand that the options will be available after a user action)

Timer displays “Done” at the end of the countdown or when the user presses the M1 Record Button again.
3. Sound file (107) icon displayed with:
   a web-enabled link (108) (which the user can just copy and paste into a telemedicine session, email or text message). The term ‘Telemedicine’ refers to any telemedicine system such as Teladoc™, American Well™ including consumer video conferencing such as but not limited to Facetime™, Zoom™ etc.
   a Play button to review/erase (119) the recording and go back to #2 and
   a Send button (not shown) to send a web-enabled link (108) to the sound(s) way file.
4. User Presses Send Button

A window opens showing additional fields for the user to add (for example):
The doctor's (103) email address (the user id unlikely to have the doctor's phone number, but this could be an additional field),
The user's name, and user's email. (The patient's information is required here so that the doctor knows they have received a link from a specific patient e.g. John Smith. Also required when multiple users use the same device to help Medaica know where to store data and create different user pages.).
If the user is sending the link via an email, the user may need to add a unique username (if they have not already) and their email (in case the doctor needs to communicate with them). If the user has already added a name or email, then the system will remember that name (via the UDID) and could provide prompts to edit that name/email, add more details, or associate a new file with a new user if being used by multiple users on same device e.g. a family, which the system could confirm when it sees different user names against the same UDID.

In another embodiment, the user might have a unique secure name that only the doctor or the doctor's system knows (such as but not limited to a patient record number, enabling the patient to exchange details without the Medaica website having the identity of the patient).

In yet another embodiment, the system could enable a blockchain feature that further secures the patient's details, and would also provide the ability to set further access rights as well as provide audit trails for users to see who and when people have accessed their details. In such an embodiment, a “heath wallet/pass” would enable the patient to be the secure owner of their own heath data, providing not only access to it, but also controlling who, where and when they give such access, and enabling full auditable data if they (or other parties) need proof of info/access.

If the user selects Send without adding their minimum details, the system will prompt them to add an identifying name. The identifier need not be unique as the actual unique identifier is the UDID+the user name. Only if a user creates a new user with the same name will the system protest.

The system can further require the user to confirm if they are the ONLY user of the device, thereby enabling the system to associate a new or different users with device (e.g. family members using same device) AND a user using more than one device.

Optionally, the SEND window could also have options for a receipt checkbox. Selecting the receipt checkbox enables the user to get a notification that the file has been reviewed (this gives Medaica another chance to get the user's email address and can also give additional trust to the user that their file has been accessed by the Doctor and/or not accessed by others).

Optionally, the web-enabled link could have features (like some URL shorteners) that limit the number of times it can be used or expiry time. This gives Medaica opportunities for example for the doctor to forward the same web link to another doctor as a premium feature or the user to limit multiple access and to have the file “expire” for additional security.
5. The doctor (103) receives either;
   a templated email/text from the user via medaica.com containing the web-enabled link to the patient's sound(s) file which contains the embedded UDID and the patient's name (or other method of identifying the patient) and email OR
   a web-enabled link (108) in their telemedicine session, pasted in by the user.

The Doctor could also receive a direct email/text from the user with the web-enabled link which behaves the same way as the web-enabled link in the Telemedicine session.)

Whether in the telemedicine session (109), text or email, the web-enabled link takes the doctor directly to the sound(s) file webpage (106) where he/she can listen to the file.

The system can also have an option of generating a web-enabled embed code which, when pasted into the telemedicine system, displays the Medaica “player” with the sound (or other) file(s). In such an embodiment, telemedicine systems could enable the doctor to review the sounds and/or perform a virtual exam without leaving the telemedicine website.

Alternatively, the system might only grant access to the file in a compressed format which would typically be good enough (e.g. CD quality) for most professional use.

However, the uncompressed (RAW) file could be more useful to certain users and applications, for example, for machine learning, AI or other research functions, in which case, that file could be made accessible to authenticated users via their access rights.

Alternatively, with the web-enabled link, having been sent by the user, there is an implicit permission from the user to the doctor to access their file, and a risk of anyone else reviewing that file does not risk leaking private data, as only the user's sound file is accessible.

### Use Case 2—Live Stream/Virtual Exam (See FIGS. 6 to 10)

A virtual exam is typically initiated by the doctor (rationale: otherwise the doctor would be waiting for the user, which is not only less efficient for doctors, but also for the user), via their telemedicine platform of choice (109) and does not require any additional tools or software within their telemedicine platform to operate.

The user (100) has simple instructions from multiple channels; a) medaica.com b) M1 device and c) if M1 was sent to them via Telemedicine Platform text/email.
1. The doctor (103) visits medaica.com (106) and clicks on the ‘clinician's tab’ and can either: click a secure/temporary pass or enter his/her login/password details.
2. Within the clinician's tab, the doctor selects “Exam Room” The Exam Room displays two fields: a room code with a <6> figure random number and a blank ‘Doctors’ Invite' code field.

The Exam Room could display reminder text re the patient: e.g. “Ask your Patient to follow these 3 easy steps 1) Plug in their M1, 2) visit medaica.com then 3) Enter the 6 figure Exam Room Code under the Exam Room tab. When your Patient does that, they will get a Doctor Invite Code for you.”
3. The patient accesses medaica.com and clicks on the Exam Room tab

The patient see two blank fields, an Exam Room field and a Doctor's Invite field.
4. Patient types in the Exam Room number given by their Doctor.
5. The Doctor Invite Code field then displays a <6> figure random number which the patient tells the doctor. Once the doctor types the invite code into his/her screen, the doctor and the patient are in same Exam Room.

The doctor can now listen live to M1 (ideally through high quality headphones (110) connected via either wireless (112) or wired (113) such that he/she can hear lower frequency sounds) and guide the patient accordingly. The doctor's headphones (110) can also be a suitable electronic stethoscope, capable of listening to recorded files on a web-enabled device.
6. If the doctor wishes to record the sounds from ‘Livestream Mode’, he/she can select a ‘Record’ function.
   We now list some further features:
1. Other DMDs including other digital stethoscopes, but also devices that record medically-related audio, image or video or other media types that would typically require interpretation by a healthcare professional, can send their files to the Medaica website. These files are then able to be accessed by healthcare professionals using the same web-link (i.e. web-enabled link) methods described. The advantage of doing this for the DMD provider is that they do not need to separately integrate their devices into a telemedicine system and the advantage for the healthcare professional is that they can now use multiple DMDs within their chosen telemedicine system.
2. Related to #1, in an Internet of Things (IoT) scenario, it is envisaged that multiple devices will be able to constantly monitor events such as laboured breathing, a baby stopping breathing, a patient's cough etc. This type of background monitoring is similar to what a device such as Amazon's Alexa does when it is constantly listening for a user's key commands. In the IoT scenario, these devices, once they detect a potential health-related issue, can then send the files (such as but not limited to an audio file), together with some device and/or patient information, to the Medaica website, where a healthcare professional can review the files to decide if further action is required.
3. It will be appreciated by those skilled in the art, that once such a system has sufficient market acceptance, it can also act as a central hub for research and other related services. Examples include 3^{rd} party diagnosis (which could be via human or machine techniques), medical insurance intelligence (which can evaluate macro trends to fine-tune their products and services). For example, looking at some or all heart-related conditions in a specific geographic location, over a specific age group, over a period of time, could help reveal trends that could be used to pre-emptively prevent patients needing more acute care.
4. The idea of a data avatar is presented to help interested parties (such as but not limited to researchers, insurance providers etc) generate a generic patient, from otherwise private pieces of data. By doing this, the recipient of the data avatar need not know that they have specific data about a patient, rather they have pieces taken from perhaps hundreds, thousands or millions of patients, to create the “typical” patient to be reviewed. The system generating such a data avatar can therefore serve the recipient without the recipient needing to browse through more complex database structures. The resulting file could also contain information that it has data from x number of patients in each of the query categories, which could further give a degree of confidence to the recipient. It is further understood that the cost of conducting clinical studies and/or other patient-related studies can be expensive and slow, so such a system could provide a dramatic advantage to the recipient. Furthermore, such a system could not only provide a specific output (the data avatar) but could be configured to require a specific “health query language” as an input to query anonymous bulk user data. This would not only enable the system to provide the appropriate results, but also standardize how multiple users, vendors and models can be uniformly addressed. There is further potential for such a system to prevent exposure of private data (under HIPAA or GDPR or similar) to outside parties and yet provide compliant/secure results.
5. In another embodiment, such as system could also provide reputational data to patients (or other interested parties). For example if a file is reviewed by a 3^{rd} party for a doctor or patient, the system can know that the reviewer has reviewed x files and achieved an accuracy rate of x % (determined by the number of times other reviewers have agreed or disagreed with the first reviewer or other such techniques). Whist such methods are known in social media (for example, a product review can display the reviewers record of reviewing products, an Uber driver has a reputational score built from multiple rides etc), these techniques have not been used or able to be provided in healthcare. By providing a system that is not only agnostic to devices and telemedicine system, but also can support patients being able to use the system in “guest mode” and providing data avatars, the system is predisposed to being a more trusted interface for all users.
6. In a further embodiment, the website and/or application provides a method of helping the patient correctly position the DMD by providing an Augmented Reality (AR) composite video of the patient and the device. The device is recognised either by its unique shape or a code (or other recognised methods) that the camera can identify. The system traces the outline of the patient and, with the identified DMD, can now direct the patient to move the device to a desired position on the patient. Such a system has additional advantages for educational purposes.
7. In another embodiment, the user sees an outline of a human torso in the video feed, in which the user best positions him/herself. The outline also displays an auscultation target icon. The user moves the stethoscope head to be within the auscultation target and can then start recording the auscultation sound. Similarly, this embodiment can be leveraged by the healthcare professional on the other side of the video feed, by moving the auscultation target to sites that he/she desires to listen to. Furthermore, these sites can be tagged alongside the recordings to aid either store and forward diagnose or archive notes, as each recording will display the target location on the patient's body where it was captured.
8. In yet another embodiment, the user has the option of a bulk recording then upload function—scenario: nurses or doctors travelling around collecting sample files, then uploading multiple files once they get back online.

The interconnected web-app may guide the user to perform a number of examinations, such as:
Self-examination for the heart, via a number of auscultation (body sound) positions on the chest.
Self-examination for the lungs (front), via a number of auscultation positions on the chest and 2 on the side.
Assisted examination, via a number of on the chest, on the side and on the back.
Live examination by a healthcare professional.

Self-examinations and assisted examinations can be done at any time, recording body sounds such as heart and/or lung sounds and then sending those results to a healthcare professional.

Alternatively, the M1 digital stethoscope can be used during a live telehealth session with a healthcare professional listening to heart and lung sounds live, guiding the user, and being able to record auscultation data together with any notes in their electronic medical records, subject to HIPAA compliant permission. This type of examination is called a live examination.

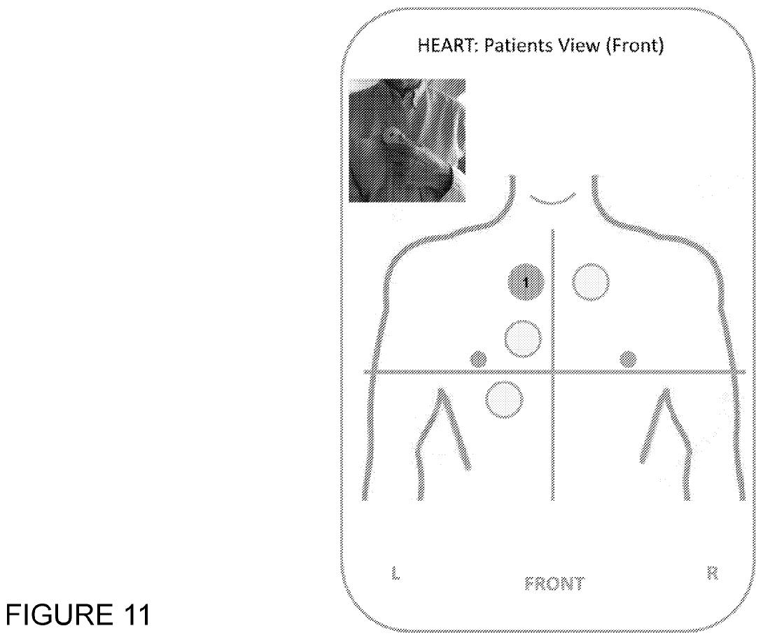

FIG. 11 shows an example of a patient's web-app displaying a mirrored view of an outline of a torso along with a video feed. In this example, the body map is mirrored for interfaces for self-examinations, using a mobile or desktop screen and/or camera for assistance. It will be appreciated that some embodiments of this invention do not require a mirrored version of the body map. The outline of the torso may also be displayed together with guidelines to help the patient find a specific position to place the digital medical device. The current position of the digital medical device (1) may be displayed alongside previous auscultation positions for which measurements or patient data has been generated. The next sequence of auscultation positions needed may also be displayed, either from a pre-programmed sequence or from the direct guidance of a healthcare professional. The auscultation sites can be moved by the healthcare professional in real time. Each location can be recorded alongside the audio file as tagged references to further assist in diagnosis and records.

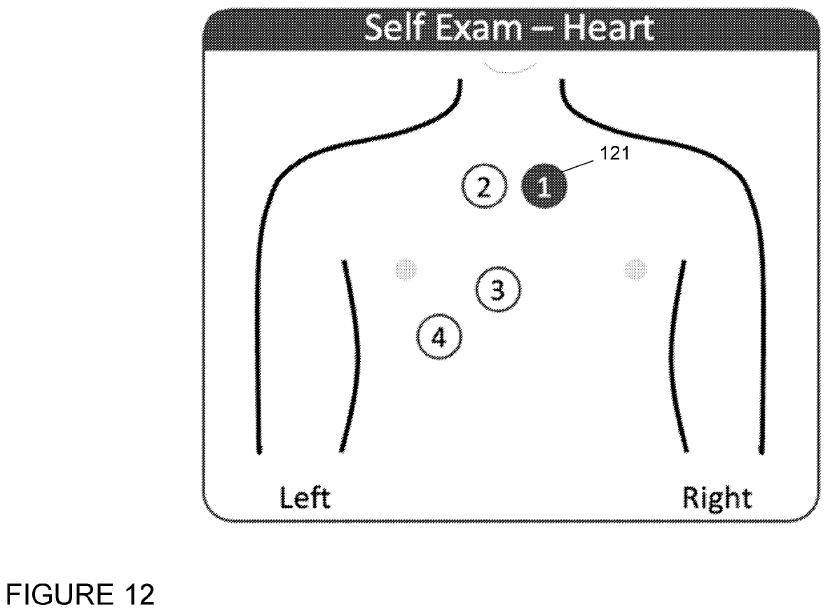

FIG. 12 shows a further example of a patient's web-app displaying a self-examination heart mode including a mirrored body map and auscultation (body sound) positions on the chest. In this example, the body map is mirrored for interfaces for self-examinations, using a mobile or desktop screen and/or camera for assistance. It will be appreciated that some embodiments of this invention do not require a mirrored version of the body map. The self-examination displays auscultation positions that a user should be able to reach without assistance. Optionally, the user is also able to select a required assisted examination option. In this example, when selecting a self-examination, a body map shows the body sound (auscultation) positions as if the user was looking in a mirror. Each auscultation position is shown as a numbered circle with the current position to be recorded highlighted, such as the first position.

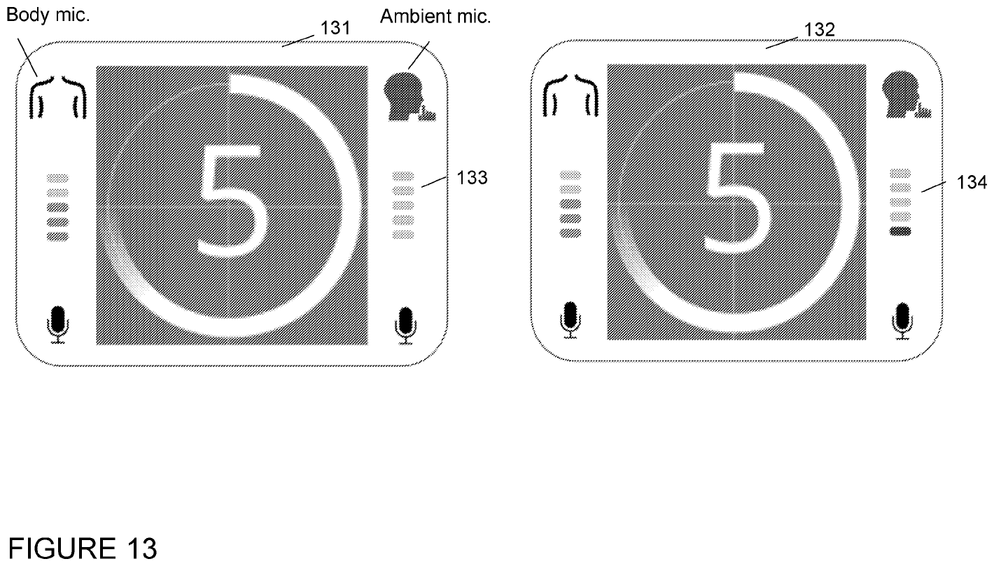

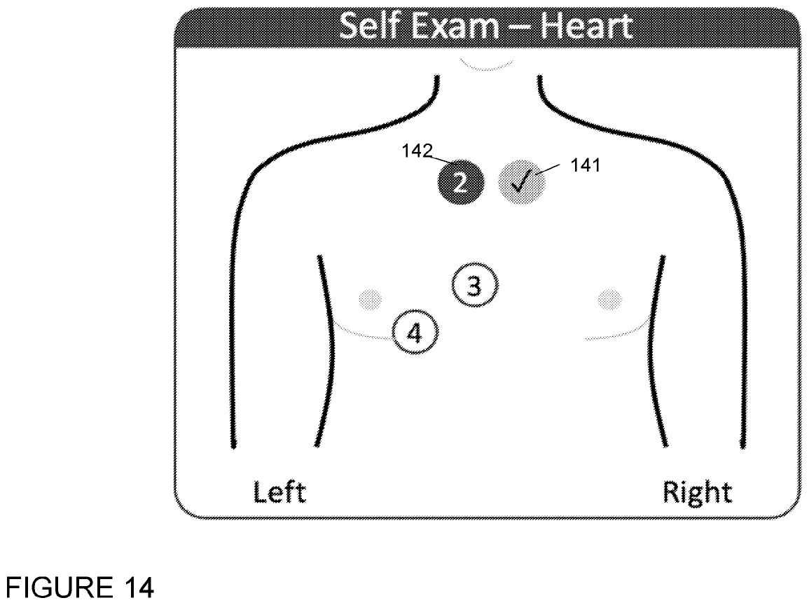

An example of the self-examination heart procedure guidance for a user using a digital stethoscope is now described:
A graphical representation of the specific examination procedure is displayed. It displays a torso outline including a sequence of required auscultation positions. The torso graphical representation is configured to guide the patient to use the digital stethoscope M1 at the required auscultation positions for a specific duration and frequency.
Place the M1 on the highlighted auscultation position (121), either directly on your skin or over light clothing such as a shirt.
Press either the M1 Start button or the Start button on the screen. Make sure there is no background noise, do not talk and do not move the stethoscope during recording. The M1 LED and the highlighted auscultation circle (121) will flash for 20 seconds indicating recording is in progress.
During auscultation recording, a countdown and recording quality window (See FIG. 13) displays the level of the recording of body sounds in relation to external ambient sounds. The level of sound received by the body microphone and the level of sound received by the ambient microphone are graphically represented. The sound level detected by the microphones is also associated with a specific color. As an example, in 131, the ambient noise displayed on the right of the countdown (133) is grey and indicates no ambient noise. In 132, the ambient noise (134) is displayed in red indicating that it is too loud to achieve a good auscultation recording. If the external sounds are too loud for a good auscultation recording, the recording will stop and a “silence” icon will be displayed.
As seen in FIG. 14, the mirrored torso outline shows when each auscultation position is recorded successfully. In this example, the body map is mirrored for interfaces for self-examinations, using a mobile or desktop screen and/or camera for assistance. It will be appreciated that some embodiments of this invention do not require a mirrored version of the body map. For example, the previously recorded position turns a different colour, such as green and displays a “tick” (141). The next recording position is then indicated (142).
The graphical representation is then configured to indicate when the exam is complete. As an example, all completed auscultation positions are displayed green.
The results can then be sent as a file to a healthcare professional by selecting SEND. The user will get notified once the exam has been reviewed. This can be an instant notification when the healthcare professional has opened and closed the file, or it can be an email confirmation sent to the user including any remarks from the healthcare professional.

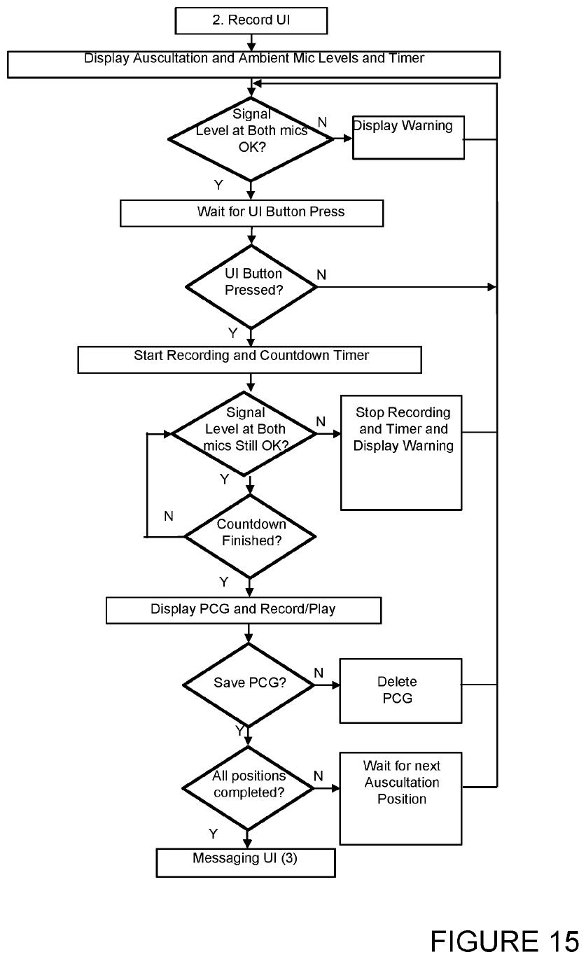

FIG. 15 is a flow diagram summarizing the steps of the self-examination procedure for recording phonocardiograms (PCG) from different auscultation positions using a digital stethoscope.

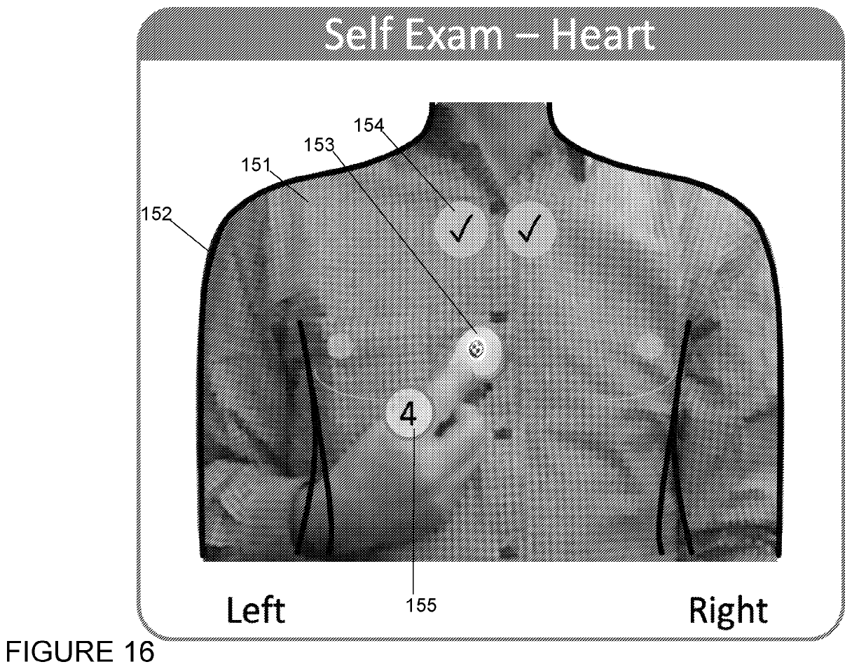

FIG. 16 shows a graphical representation of the specific examination procedure overlaid over a live video image of the user (151). The live feed of the user may include the body shown as transparent or semi-transparent, with the rest of the image masked, opaque or solid to avoid the background interfering with the live video image of the user. A torso outline is displayed (152) alongside the current auscultation position of the digital stethoscope (153) and specific auscultation positions (154,155) required by the exam procedure. In this example, the body map is mirrored for interfaces for self-examinations, using a mobile or desktop screen and/or camera for assistance. It will be appreciated that some embodiments of this invention do not require a mirrored version of the body map. The user positions him/herself inside the torso outline and can then accurately position the M1 over the required auscultation position. The current auscultation position can flash on/off so that when the M1 is in position covered by the circle it is not a confusing image for the user.

Additionally, the software may recognize a symbol on the M1 head and when the user moves M1 to the correct position, the software can prompt the user accordingly (and/or autostart recording). This can be implemented together with augmented reality techniques.

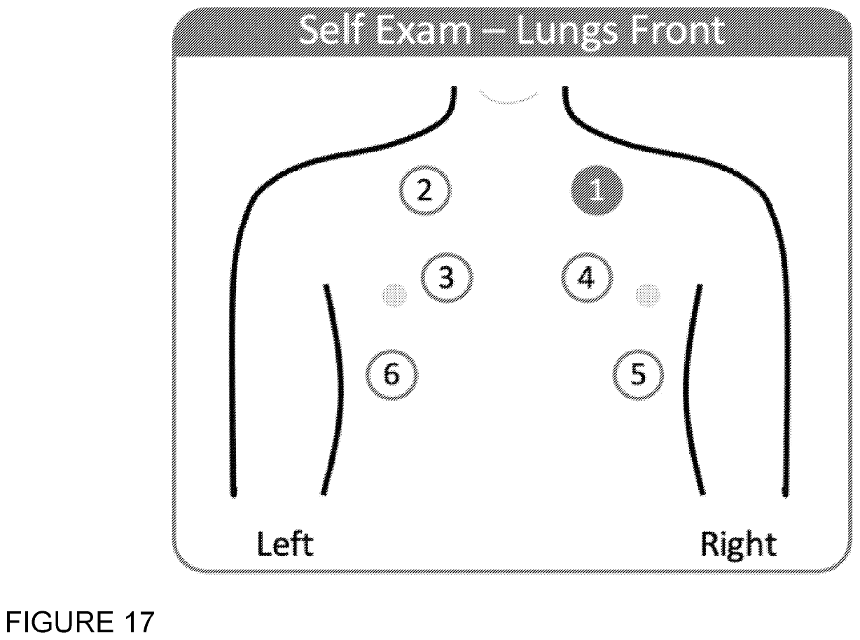

FIG. 17 shows a graphical interface of front lungs self-examination including a mirrored image of a torso outline of a front torso and required examination positions. For lung sound recording, two full deep, slow breaths should be captured.

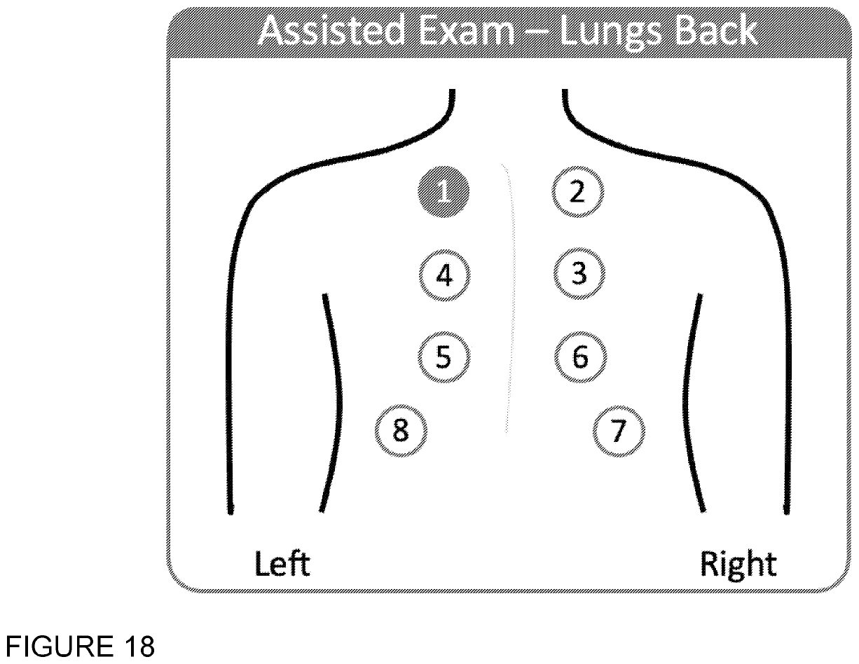

FIG. 18 shows a graphical interface of back lungs assisted-examination including a torso outline of a back torso and required examination positions.

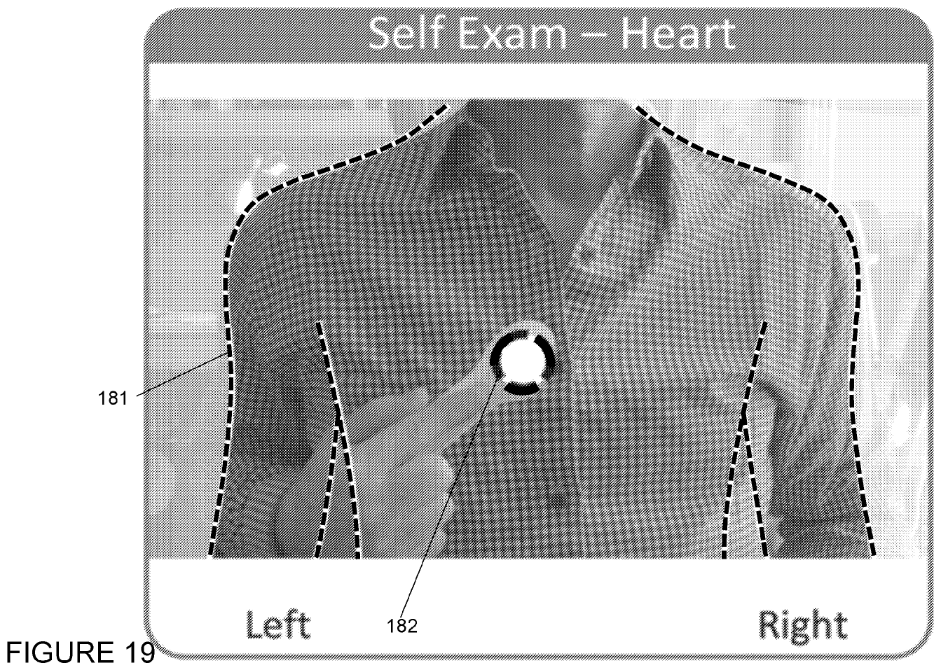

FIG. 19 shows a graphical interface of a video-positioning mode. Selecting ‘Video Positioning’ mode first displays a window asking for permission to use the video camera. For privacy, video-positioning mode is only used for guiding recording positions without recording any video. With video mode positioning on, the mirrored live video feed of the user is displayed alongside outline of the body (181) and the current auscultation position displayed as a flashing circle (182). The auscultation icon might need to alternatively flash black/white (or other contrasting colors) to make sure that whatever the user is wearing is not confusing the image. The torso outline may also need to have a black/white stroke to make sure it is visible. When the user positions himself inside the body map and holds M1 at the flashing auscultation position, recording is started when the user pushes either a start button on the digital stethoscope or an icon or symbol on the graphical interface.

### Other Features Include

The countdown/record window is automatically displayed (or pops up), such as when M1 is in position and the user is still and quiet.
A symbol on the M1 head is recognized by the image processing software and when the user moves M1 to the correct position, the software prompts the user accordingly and/or auto-starts recording.
The camera detects an outline of the user and creates a specific body map. This is done by accessing a library of auscultation positions to fit specific body types, or by re-calculating the positions based on the detected outline and specific exam positions.
The user is able to select a body map based on nearest fit.
The software automatically selects the nearest fit body map from a library based on the video feed of the user.

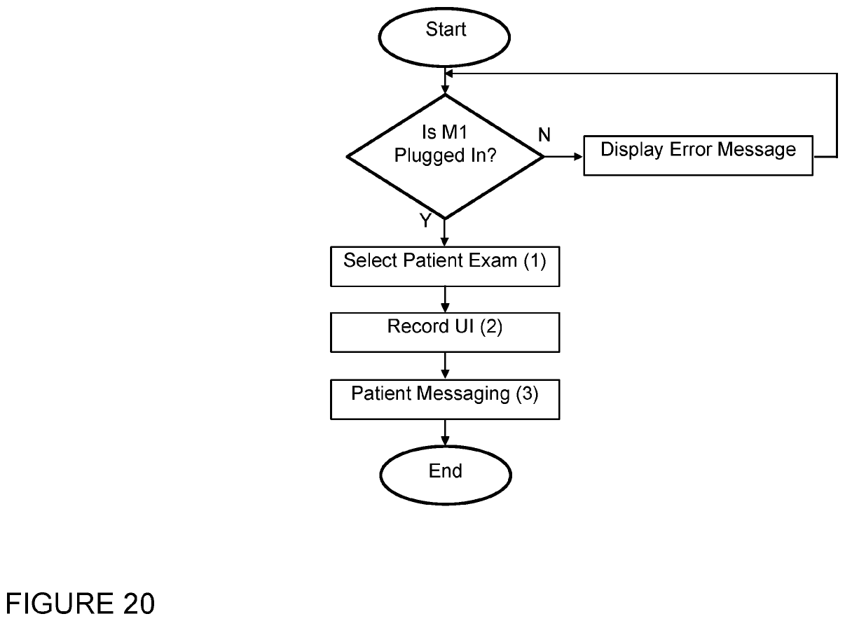

For a live exam, a healthcare professional may send the user a link to a virtual room, such as by email or via a text message or any other messaging application. Clicking the link will take the user directly to the virtual exam room. As shown in the flow diagram of FIG. 20, if the M1 device is not plugged in, or not recognized by the system, an on-screen message will be prompted such as “plug in your M1 device”. When the healthcare professional is present, the virtual room exam displays his/her name. The healthcare professional then guides the user through the auscultation positions, or moves the auscultation positions to where he/she wants to listen. The healthcare professional is able to control when the M1 starts recording each body sound.

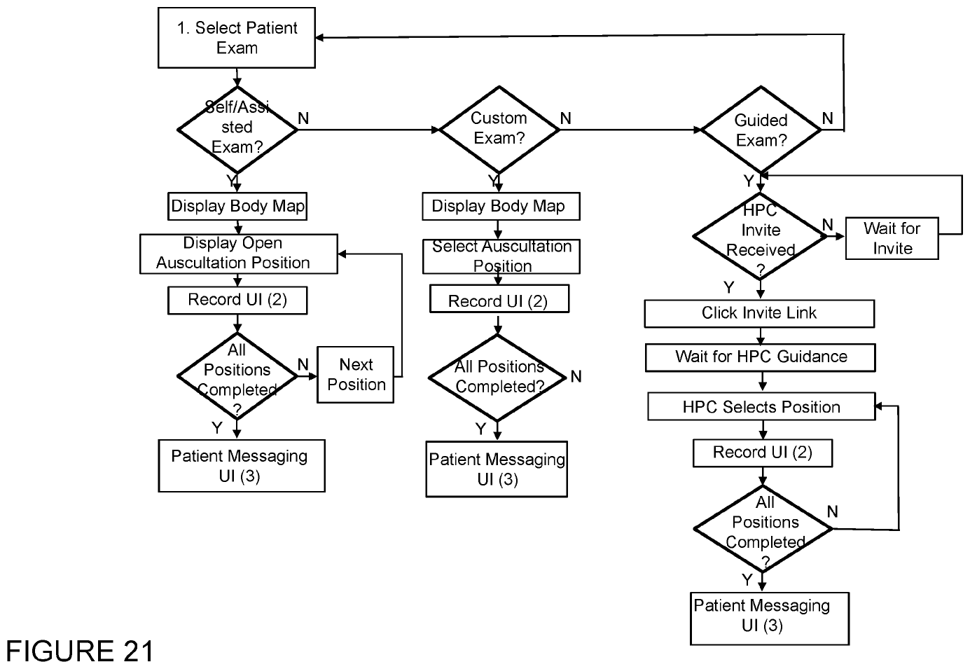

FIG. 21 shows a flow diagram summarising the different steps according to a self-examination mode, custom examination mode or guided examination mode.

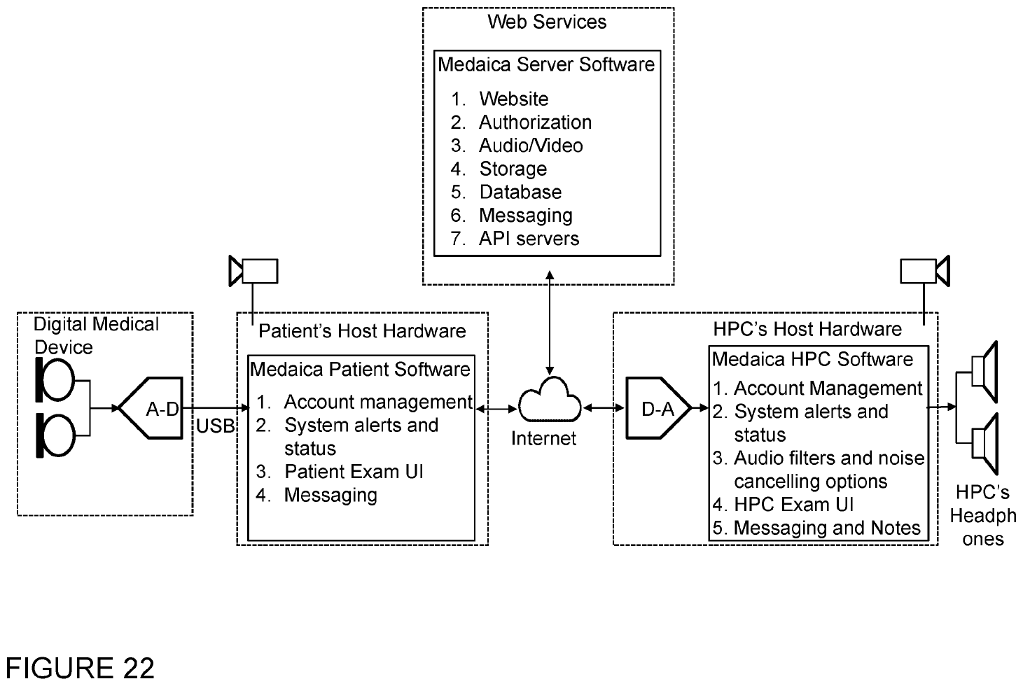

FIG. 22 shows a diagram summarizing key elements of the system.

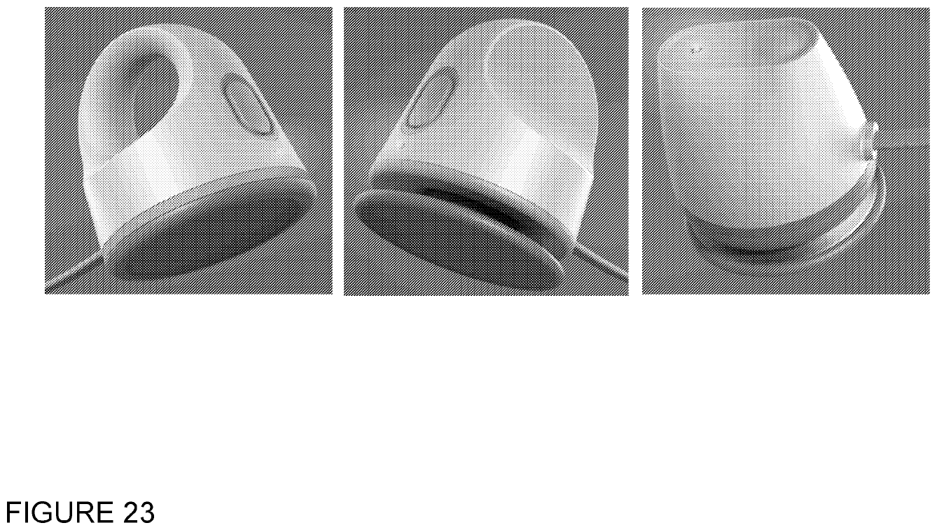

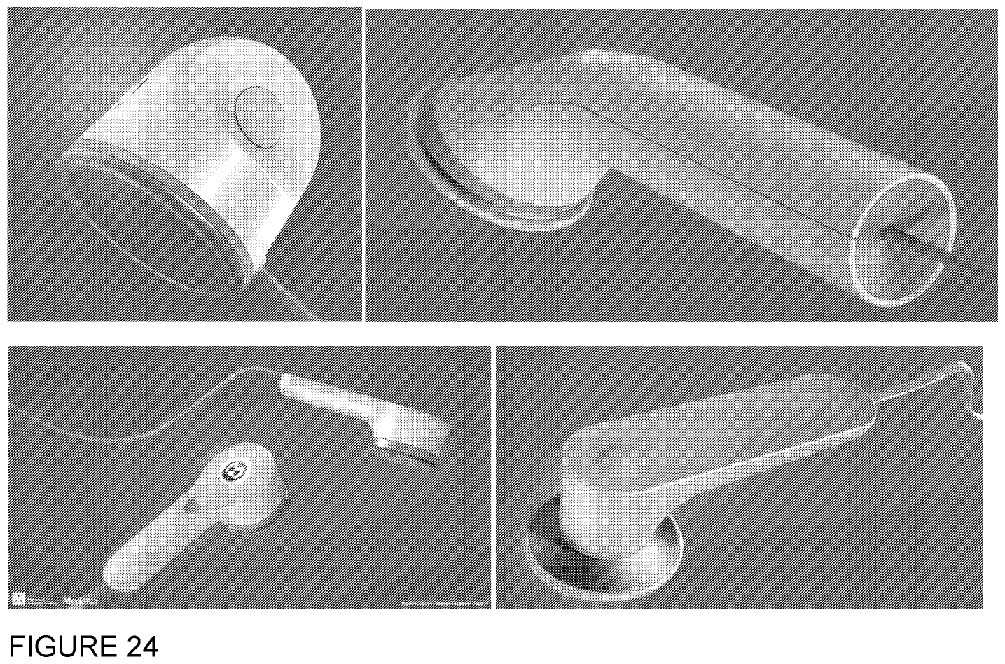

FIGS. 23 and 24 shows photographs illustrating a number of digital stethoscope devices. The designs are user-friendly, easy to grip and include at least one button.

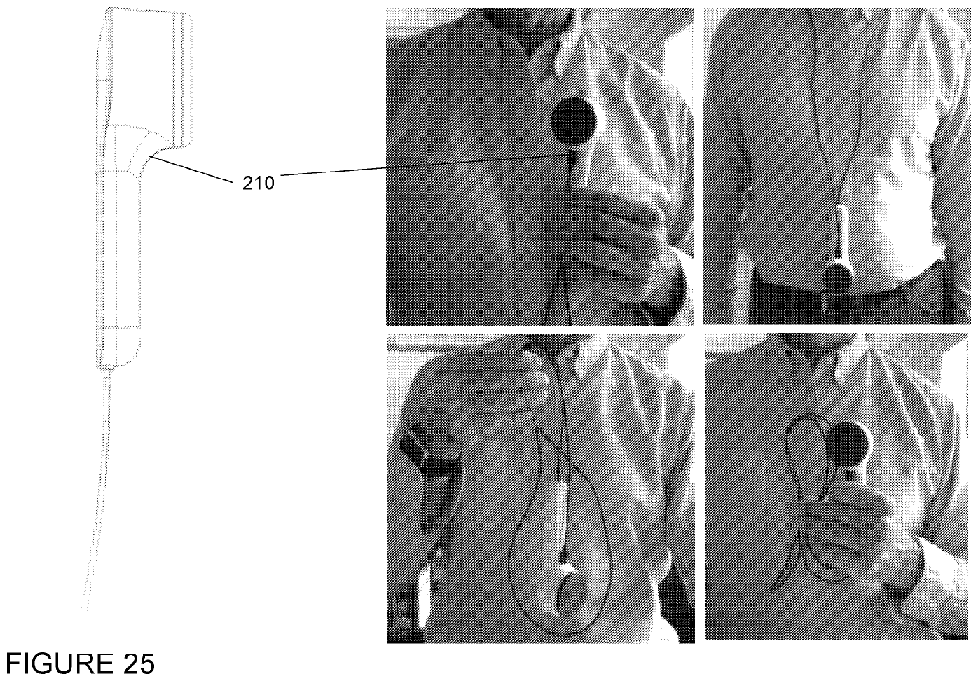

As shown in FIG. 25, the cable plug can be inserted into a dummy socket (210) in the unit to fold the cable in half when the device is unplugged. This makes the cable much less unwieldy, and easier to stow in a bag.

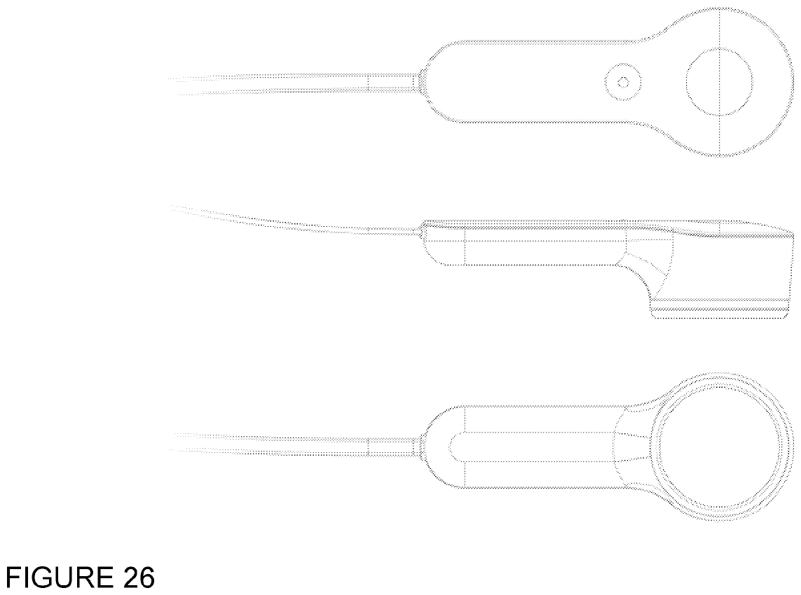

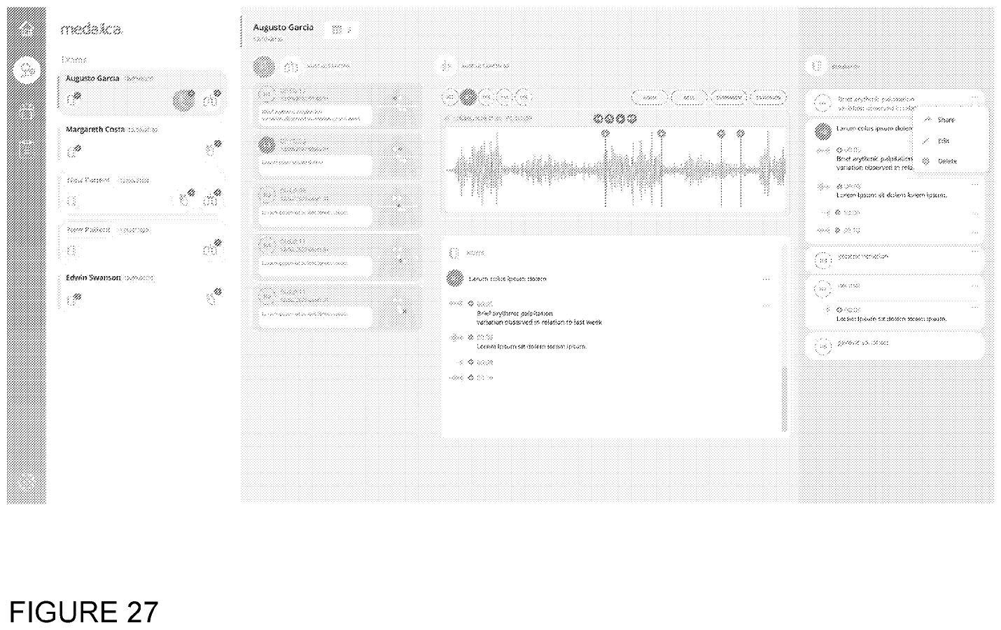

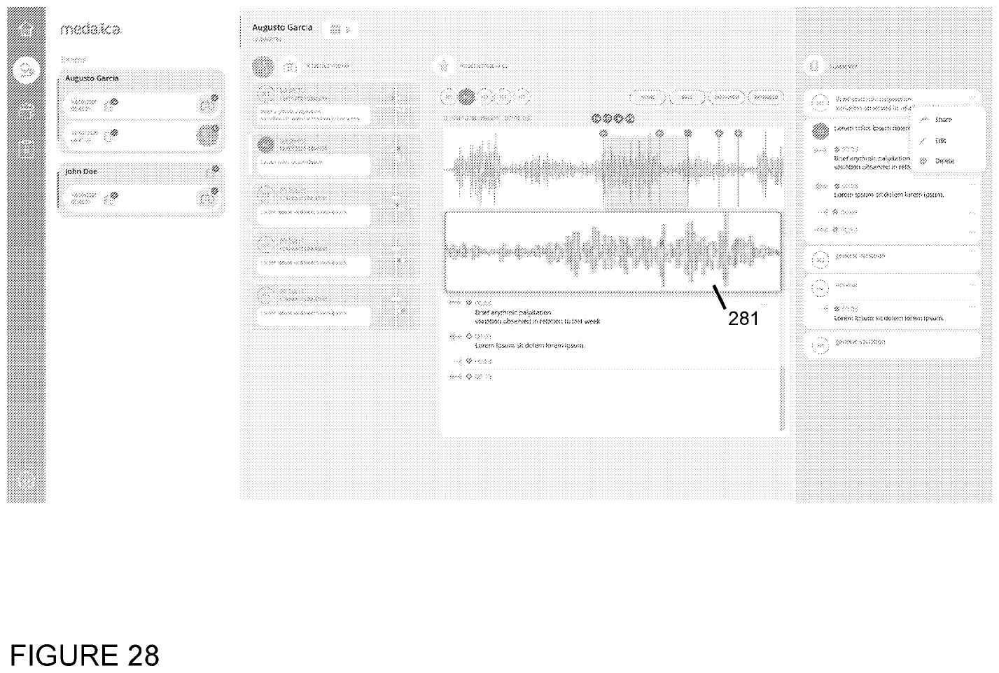

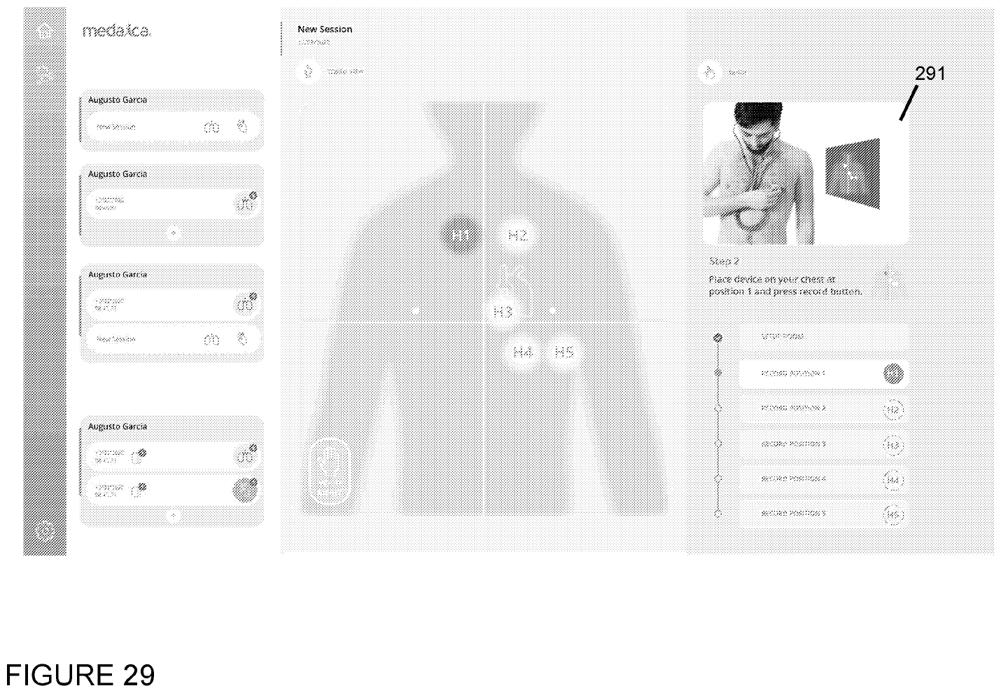

FIG. 26 shows top, side and bottom views of another example of a digital stethoscope device.

### High Level Healthcare Programming Environment

Instructions, devices and notifications can be “chained” together to help patients perform specific healthcare management protocols. For example, rather than a patient taking a general reading such as recording a heart or lung sound, the system can guide the patient to take specific tests with a specific frequency and can optionally send reminders to the patient as well as updates to the patient's healthcare provider(s) and/or insurer or other parties with appropriate permissions. For example such a system could guide the patient to use a digital stethoscope to “record heart sounds in Position 3, twice a day, for seven days”. In such an example, Position 3 could be a specific instruction with a diagram or video. That specific instruction, frequency and duration can have notifications such that user is sent reminders, and the healthcare provider is sent results.

One example of the value of such a system: a hospital could for example, set up a “Patient Release Protocol” as a one click “applet” (sending the patient a link to the applet so the Doctor will know if/when the patient is following the release procedure and recovering on plan). Such an “applet” could be different for each healthcare provider, patient and/or condition and could provide methods for the healthcare provider to brand the experience as well as integrate the outputs into their healthcare records.

When new devices, instruction modules or features are added to such as system, it adds utility to its users. For example, a patient could be taking their temperature, heart sounds recording the data for the doctor in a fairly automatic and regimented method. 3rd parties could develop simple branded applets. Applets could also be protocols for clinical trials and/or other useful applications.

### Telemedicine Device Including a Second ‘Room’ or ‘Patient’ Microphone

Adding a second ‘room’ or ‘patient’ microphone (mic) to a telemedicine device allows the patient to continue to communicate with their healthcare provider. As browser security models only allows for a single audio device to be used at any given time, it is, in the prior art, necessary to switch the audio source in the browser. For example, if the patient is on a laptop and using its default mic, they would have to switch the browser audio source to the telemedicine device to perform an exam that required a digital stethoscope microphone. This would cause the user to lose the connection with the built in mic and their means of verbal communication with their healthcare provider. Adding a second ‘room’ or ‘patient’ mic to such a telemedicine device enables the patient and healthcare provider to maintain communications and still capture exam sounds.

Initially, the audio will be delivered over a stereo channel but the web app will separate the audio signal into two separate mono feeds and will process each differently. The auscultation sound channel will have a gain control so a strong enough signal will be captured for the body recording. In addition, filters such as a low pass filter (or any other processing) may be applied to the sound (typically after the sound has been recorded, maintaining the raw audio file).

The room channel may also have a gain control but will mainly just be passed on to the room and ultimately the healthcare professional's headphones. The healthcare professional and/or patient can have control of muting each channel separately if they want to only hear one or the other mic.

In addition, the room mic can be used to capture audio that can be used to reduce or remove non-heartbeat sounds in the heartbeat audio file using standard noise reduction techniques. This specific feature can additionally be used by the system to determine if the room is too noisy for a patient reading and/or if a patient is speaking when the exam is being recorded. This information can then enable the system to display a message to the patient to be silent and/or there is too much noise to perform the exam.

An audio signal may be used to enable the capture, transmission, storage, and display of data from one or more sensors over a regular USB audio channel. This connection can work in any device that allows a microphone to connect and transmit data to a computer, phone, tablet, etc. The captured data is converted to audio using a predefined system that maps character data to audio frequency bands. Each character (number, letter, or symbol of the digital message) is mapped to a specific, unique frequency band (or mix of frequencies, like DTMF encoding, dual tone multi frequency encoding). In addition, a special “start” and “end” identifier is given a specific, unique frequency band or mix of frequencies as well (and a check sum could be added to ensure that the system has successfully transmitted the data). A set duration is established for all characters of the message so that each tone lasts the same duration. In one method, a sine wave is generated at the specific frequency in the middle of the character's frequency band that matches the current character of the message. Each message starts by sending a “begin” tone at the predefined “begin” frequency for the predefined duration. This is followed by each character's predefined frequency again at the specified duration. When complete, an “end” tone is sent to complete the message. This loops and continuously updates the message frequencies each time as the data changes. This signal is transmitted over the USB connection as regular audio and re-encoded in the browser as digital data using the same frequency band to character map. This converted data can then be captured, stored, manipulated, displayed to the user, etc. as regular digital data.

In another embodiment, the system adds a camera with OCR software to translate any digital readout (for example a blood pressure display) into audio.

Using these methods, such a system can leverage a single mono track in the stereo audio signal of a web video interface and keep a room mic open as well so patients can still talk to their healthcare provider while using and/or transmitting data from the medical device. This allows integration with any platform that either accepts an audio connection or has a display that can be read by an OCR reader and audio converted.

In the following section, we provide more detail on various details and features of the Medaica system.

### Telemedicine

As a preliminary point, the terms ‘telemedicine’ and ‘telehealth’ are often used interchangeably in the public domain; Medaica follows that approach. Telemedicine is a subset of telehealth that refers solely to the provision of health care services over audio, video and/or messaging platforms via mobile phones and/or computers. Telemedicine involves the use of telecommunications systems and software to provide clinical services to patients without an in-person visit. Telemedicine technology is frequently used for follow-up visits, management of chronic conditions, medication management, specialist consultation and a host of other clinical services that can be provided remotely. Furthermore, the WHO also uses the term “telematics” as a “a composite term for both telemedicine and telehealth, or any health-related activities carried out over distance by means of information communication technologies.”

It is also noted that some high-end telemedicine systems, typically used by hospitals for follow-up visits, often require complex and expensive “medical carts”, operated by skilled doctors, nurses or technicians at the patient's location connecting to operators at a medical center. As is often the way with technology advancements, many companies are now providing some or all of these features to doctors and/or patients via more affordable devices and/or smartphones. There is however, an increasing requirement to address ease-of-use, scalability and security as such systems start to gain wider appeal.

For the purpose of this document, the term ‘telemedicine’ should be broadly construed to encompass telehealth and telematics, and is not limited to professional or consumer systems.

Additionally, the terms ‘doctor’, ‘healthcare professional’, and ‘clinician’ are interchangeable and may also refer to nurses or any other practitioners who might not be doctors.

### Auscultation Hub

The Medaica ‘Auscultation hub’ is a website that stores files, such as but not limited to auscultation recordings from users' devices such as digital stethoscopes. The auscultation hub enables easy linking of those recordings to/from health practitioners and telemedicine platforms. The auscultation hub also enables editing of auscultation audio files; for example, a source audio file could be a sound recording lasting 60 seconds or more. But that sound recording could include extraneous noises of no clinical significance; the doctor/healthcare professional can review that complete auscultation audio file from within the auscultation hub and edit out or select sections of clinical relevance; the edited sound recording can be shared, for example with experts for an expert opinion, by sending that expert a weblink that, when selected, opens a website (e.g. the Medaica Auscultation hub) and the expert can then play back the edited sound recording.

### Virtual Exam Room

The Medaica ‘Virtual exam room’ enables a doctor/healthcare professional to send a web-enabled link to patients as an invite for a virtual exam that will take place in the Medaica virtual exam room. A patient clicking on the web-enabled link is taken to a webpage virtual exam room, which display instructions which could include timing for exam, instructions to be ready to place the stethoscope where the doctor requires it etc.

The exam session can be recorded and data files sent to 3rd parties to review/diagnose. The doctor/healthcare professional can also edit files and send edited files to other experts, as described in the Ausculation hub section above. In some implementations, the doctor can initiate the record start/stop from the website (i.e. not requiring the patient to initiate from the device).

### Web-Enabled Links to/from Auscultation Files and/or Other Medical Records

Files/records are not sent to doctors or telemedicine systems. Instead, the Medaica system generates a secure and unique web-enabled link or web link that, when clicked on, takes the recipient to that file. The unique web-enabled link can include meta data such as but not limited to date, time device ID, user info, but also, if there are business model rules such as but not limited to access rights, permissions, number of clicks per link permitted, rate per click, billing codes etc.

The web link could also have a one-time or multiple use feature which could in turn be linked to the user's membership rights (as could any of the aforementioned features).

Access rights could be leveraged to subsidize the business model e.g. assuming access options include telemedicine platforms, insurers, research etc. and, if research is enabled, the session could be free to patients if they agree to the terms that their data is being used for research and/or is being supported by a charity, e.g. the Gates Foundation.

Related is that the web link could also offer a drop-down menu to compatible telemedicine systems and/or doctors nearby etc. Referral programs could then support Medaica when Medaica customers link to a specific telemedicine platform.

The system can also have an option of generating a web-enabled embed code which, when pasted into the telemedicine system, displays the Medaica “player” with the sound (or other) file(s). In such an embodiment, telemedicine systems could enable the doctor to review the sounds and/or perform a virtual exam without leaving the telemedicine website.

### Security/Permissions

Users can have certain rights to listen, review, tag, annotate, forward, analyze, download files. For example, if a doctor does not have permission, he/she cannot tag the file with an opinion. Similarly, a 3rd party could be supported to give an opinion of the file, but not have permission to re-send the link. (If they cut and pasted the link they received, the system would know it was a one-time review link and would have expired and the system would inform the system owner/user/admin of the attempted impermissible use.

### Watermarks

Sound files can be watermarked such that if they are downloaded or used off-site, it can be easily determined that they are Medaica files. Such watermarks could be overlaid/added to Medaica files in a unique manner that the system could know how to remove alter (for example adding new date/user/owner info).

### Collaboration and Verification Features

3rd parties such as analytical labs and/or researchers, can be granted access to files, either by system admins, or by doctors or other authorized users to diagnose files and/or enable a second opinion and/or conduct research for local government or other medical research, subject to their access rights.

Researchers could also be granted access to multiple files based on time, type, region etc. 3rd parties could also provide a crowd sourced human verification diagnostic solution (like CAPTCHA) whereby x people claiming a sound is a certain condition, increases the confidence that that sound is indeed that condition. This could be further enhanced, to give doctors confidence that the diagnosis has been conducted by peers, for example by providing auditable references (e.g. clicking on who reviewed the sample—how many samples he/she has been credited with correctly reviewing etc.).

### Business Model(s)

There are numerous anticipated business models including but not limited to:
1. charging telemedicine platform providers $x for every telemedicine session that leverages a Medaica exam (determined via the web-enabled link data). This could be a revenue share of the incremental reviews generated by such traffic.
2. Telemedicine platform providers could use Medaica devices for customer acquisition—i.e. they send users a Medaica device for free or for a discount if they sign up. They would do this because with Medaica, users will be getting a more useful telemedicine session, and the platform providers will be getting higher revenue and (until Medaica is ubiquitous), a more competitive solution.
3. Medaica could sell direct to end-users (patients) with a coupon for a discount for their first telemedicine session with Company X.
4. Medaica can charge a per click or per seat fee—per click could be based on types of clicks e.g. a doctor listens to a file is standard rate, but if she/he forwards the file for diagnosis, that could be a different rate (higher or lower).
5. As mentioned under Smart/Web-enabled links, Medaica could have a third party subsidize each recording and/or click in return for the data/research potential.
6. Insurers will be interested in participating in the ecosystem if a Medaica session can help triage the need for patients to have more expensive exams or in person visits.

### Agnostic/Plug and Play

Most medical devices have proprietary systems and, in the case of digital stethoscopes, cannot easily interface with telemedicine systems. This is even more challenging with Bluetooth devices as they can compete/confuse systems and devices that assume Bluetooth is for communication with the user not a device and rarely can handle communicating with both a device and a user (in a telemedicine session, a Bluetooth stethoscope will typically take over the audio channel, making it impossible for the patient to talk or hear the doctor).

Furthermore, most telemedicine platforms are closed systems and cannot easily enable device integration. Similarly, most medical devices are closed systems and/or have their own telemedicine solutions, making them ill-suited to multiple telemedicine solutions. Even in a well-designed telemedicine system, or video platform, the ability of using an additional device will invariably require a new window or tab or menu item to be selected, so Medaica not only provides the same utility as a well-integrated solution, but does so for ANY video platform. The virtual Exam Room is simply a new window that can be clicked on outside of the telemedicine screen, but without having to launch a complex alternative application.

### Appendix 1—Key Features of the Medaica System

One implementation of this invention envisages an internet-connected app that is hardware agnostic and can hence be easily deployed across all Android and iOS smartphones; virtually any medical device can be easily and cheaply architected to send patient datasets to the smartphone, e.g. over a standard USB cable; and the internet-connected app can then manage the secure transfer of these patient datasets to a web server. Once on the datasets are stored on the web-server, they can be shared by generating a web-link to those specific datasets and sharing that web-link; any physician with a web browser can then review those datasets.

One conventional approach when designing telemedicine systems is to provide some sort of proprietary and secure data transfer system directly into the medical device or a host computer; this data transfer system can then securely transfer data to a cloud-based telemedicine system. So the architecture is quite simple: medical device connects to telemedicine system. In one implementation of this invention, the overall architecture is more complex, because we add in an internet-connected app (resident on the medical device or a connected smartphone etc.) and a web-server that the web-app communicates; that web-server can then in turn connect to the cloud-based telemedicine system.

So we have added additional layer of complexity to the overall architecture. But, paradoxically, by adding this extra complexity, we enable simplicity: This approach de-couples designers of medical devices from the complex technical challenges of the secure transmission of confidential patient data and integration into proprietary telemedicine systems: all they need to include is a standard data transfer system (e.g. USB cable) and so they can focus on doing what they do best, namely designing the best medical devices they can. Likewise, it de-couples designers of telemedicine systems from these same technical challenges: they can instead focus on doing what they do best, namely designing systems that best serve the needs of healthcare professionals and patients.

One can draw an analogy to the early days of personal computing. If you wanted to build a peripheral device, such as a laser printer, then you would need to understand and implement some sort of data transfer system that enabled you to communicate quite deeply with the computer's hardware—e.g. memory where documents were stored. This required laser printer designers to master the intricacies of how CPUs and memories operated. But then a universal abstraction layer was added—such as the Windows® operating system—this adds an additional layer of complexity to the overall architecture, but was fundamental to enabling overall simplicity: laser printer designers could simply ensure they could work with the Windows operating system and focus on doing what they did best, namely designing laser printers that will work with any computer from any manufacturer, so long as they ran on the Windows operating system.

The present invention offers the same potential to enabling medical device vendors to focus on what they do best, enabling the design of medical devices that work with any telemedicine system, so long as the medical device can include an internet-connected app or send data to a device like a smartphone etc. that can run an internet-connected app; and so long as the telemedicine system has a web browser. Similarly, it enables telemedicine vendors to focus on what they do best, without having to be concerned about the specifics of how medical devices work, or requiring medical devices to include specific proprietary software.

Since all smartphones etc. run web apps, and all telemedicine systems can use a web browser, this invention can provide a universal backbone connecting in essence any medical device to any telemedicine system. In the following sections, we outline four features of the Medaica system; we list also various optional sub-features for each feature. Note that any feature can be combined with one or more other features; any feature can be combined with any one or more sub-features (whether attributed to that feature or not) and every sub-feature can be combined with one or more other sub-features.

### Feature 1: Web-Links

In one implementation, a telemedicine system enables patient datasets that are generated from multiple medical devices to be sent to a remote web server or servers. For example, there could be thousands of low-cost stethoscopes, e.g. M1 devices as described in this document, each being used by a patient at home by being plugged into that patient's smartphone using a simple USB cable connection. Each smartphone runs an internet connected application that records the heart etc sounds captured by the tethered stethoscope and creates a dataset for each recording. It sends that recording, or patient dataset, to a remove server over the internet. The remote server then associates that recording, or patient dataset, with a unique web-link. The patient's doctor is sent the web-link, or perhaps the server sends the web-link for automatic integration into the electronic records for that patient. In any event, the patient's doctor can then simply click on the web-link and then the recording or other patient dataset is then made available—e.g. a media player could open within the doctor's browser or dedicated telemedicine application and when the doctor presses ‘play’, the sound recording is played back.

We can generalise to:

A telemedicine system comprising one or more medical devices that are each configured to generate patient datasets, and a remote web server; in which:
a medical device is configured to upload or send patient datasets to the remote web server, directly from an internet-connected app running either on the medical device or on an intermediary device;
the remote web server is configured to generate a unique web-link that is associated with a specific patient dataset;
and in which the unique web-link enables a healthcare professional to review the specific patient dataset by selecting the web-link from within a web browser or from within any dedicated telemedicine application that opens web-links.

And we can further generalise to:

A telemedicine system comprising one or more medical devices that are each configured to generate patient datasets, and a remote web server connected to at least one of the medical devices; in which:
a medical device is configured to upload or send patient datasets to the remote web server, directly from an internet-connected app running either on the device or on at least one intermediary device;
the remote web server is configured to generate a unique web-link that is associated with a specific patient dataset;
and in which the unique web-link is configured to enable a healthcare professional to review the specific patient dataset by selecting the web-link from within a web browser or from within any dedicated telemedicine application that opens web-links and to initiate a virtual examination of the patient by opening a link to a virtual examination room hosted on the remote web server.

### Optional Features:

### Remote Web Server:

the remote web server is configured for recording, storing and controlling access to uploaded patient datasets.
the remote web server posts or makes available webpages that include the patient datasets and can be viewed on any web-enabled device, such as the patient's laptop, or mobile phone or the healthcare professional's laptop or mobile phone.
there are multiple remote web servers

### Web Link:

the web link is configured to be copied and pasted by the user into a telemedicine session, email message, text message or any other communications system.
the web link is configured to be sent automatically to the healthcare professional.
the web link is configured to be sent automatically to the healthcare professional only after the user has confirmed it should be sent by interacting with a web page posted by the web server.
the web link, when selected by the healthcare professional on their web-enabled device, takes the healthcare professional directly to the patient dataset stored on the web server, to enable the healthcare professional to review that patient dataset.
the web link is configured to be used to control access rights and privacy control access rights.
the web link is configured to be used to control additional healthcare services such as diagnostic analysis and verification.
the web link contains rules permitting third party access right, sharing/viewing rules and financial controls.
the web link is a HTML hyperlink.
the web link when selected opens a video conferencing application.
the web link when selected opens a video conferencing application that is integrated within a telemedicine session
the web link provides access to the patient dataset to an authorized third party only when the authorized third party has been authenticated by the system and/or patient and/or healthcare provider.
an authorized third party accesses the patient dataset in real time as it is being created.
the method enables an authorized third party to start or stop the creation of a patient dataset by at least one of the medical devices.
the method enables an authorized third party to record the patient dataset.

### The Intermediary Device:

is a smartphone or laptop or any other computing device that is configured to connect to at least one of the medical devices and the remote web server.
the medical device connects to the intermediary device using a data cable, such as a USB cable.
the medical device connects to the intermediary device over short-range wireless, such as Bluetooth.

### The Medical Device:

the medical device is any digital medical device that can generate patient data and send that data, directly or via an intermediary device, to a remote web server.
the medical device is one of the following: digital stethoscope, ultrasound, blood pressure monitoring device or any other digital monitoring devices.
a visual indicator on the digital medical device automatically turns on when a patient dataset is being generated.
a visual indicator on the digital medical device indicates when sufficient data has been measured to generate a patient dataset.
a visual indicator on the digital medical device indicates that an authorized third party is accessing, such as streaming the patient dataset.
the medical device is a smart device that is configured to monitor vital signs and other patient parameters for anomalies or events and to automatically send an alert to the remote web-server if an anomaly or event is detected, together with a patient dataset that captures the anomaly or event, and generate a unique web-link that is associated with that patient dataset and to send that unique web-link to a healthcare professional or emergency service.
the anomaly or event includes an onset of organ failure or malfunction
the anomaly or event includes an altered breathing rate or cough
the medical device connects to the intermediary device running the web app over a USB port.

### Feature 2: Second Microphone: Telemedicine Audio Systems and Methods

Maintaining communications between a patient and healthcare professional while examination sounds are being shared is currently still a challenging task. In the example we gave above, the patient used a simple stethoscope connected via a USB-C cable to a smartphone; after the patient had completed recording his heart/lung etc. sounds, the recording was sent by the smartphone to the remote server, and a web-link was generated by the server and then sent to the patient's doctor. The doctor could hence review the patient's records a few hours or days etc. after the patient had made the recording by selecting and opening the web-link in a browser. But in the Medaica system, the doctor can start a video or audio examination of a remote patient, and during that examination can choose to listen to the real-time heart/lung sounds being recorded by the stethoscope the patient is using (using for example the web-link sharing process described above), and can also have an audio conversation with the patient because the stethoscope includes two microphones: one for picking up the heart/lung sounds, and a second microphone for picking up the voice of the patient. The doctor, when listening to heart/lung sounds, can mute those sounds fully, and instead listen to the patient talking; the doctor can also partly mute either the heart/lung sounds or the patient's voice; for example, to have the heart/lung sounds as the primary sound and have the patient's voice partly muted and hence at a lower level. Similarly, the doctor may have the patient's voice as the main sound and have the real-time heart/lung sounds muted to a lower level.

Using one microphone per channel, i.e. one microphone on the left channel and the other on the right channel, allows the design to leverage common amp and/or A-D chip designs.

Without this design, a system would need a method of switching from the auscultation/stethoscope microphone to the patient voice microphone, which is challenging to engineer since it requires a system-level change. Further, being able to process the sound signals from both microphones in parallel can be very advantageous for various noise reduction/cancellation and enhancement functions. For example, in a noisy environment (e.g. in an ambulance, ER) noise reduction/cancellation techniques can be applied such as measuring the timing/phasing of noise detected by the voice microphone compared with the same noise detected by the auscultation microphone: this requires simultaneous or parallel processing of the sonic signals from both microphones, and would not be possible if the auscultation/stethoscope could only be sending signals when the patient voice microphone was off, and vice versa.

Simultaneous or parallel processing of the sonic signals from both microphones also enables compensating for different timing in receiving auscultation sounds in patients with different body masses: for example, assume the patient voice microphone detects a sound in the room with a given intensity; that same sound will pass through the patient's upper body tissue and be reflected off the ribcage and hard tissue; the auscultation/stethoscope will detect that reflected signal. But the attenuation of the reflected signals increases as body mass increases; hence we are able to approximately infer body mass by measuring the intensity of the reflected signals; we can use that body mass estimation to compensate for the small but different time delay in receiving auscultation sounds in patients with different body masses, and can hence normalise auscultation sounds across patients in a way that compensates for different body mass.

We can generalize to:

A telemedicine system comprising: multiple medical devices that are each configured to generate patient datasets, and a remote web server connected to each medical device; in which:
a medical devices is configured to upload or send patient datasets to the remote web server, directly from an internet-connected app running either on the device or on an intermediary device;
and in which the medical device includes (i) a speech microphone configured to detect and/or record patient speech and (ii) a second microphone in the medical device configured to detect and/or record clinically relevant sounds and generate an audio dataset from those sounds;
and in which the internet-connected app is configured to treat that patient speech separately from the audio dataset and is hence configured to enable real-time voice communication from the patient to the healthcare professional at the same time as the audio dataset is being shared with the healthcare professional via the remote web server;
and the system is configured to enable the healthcare professional to select whether to listen to real-time voice communication from the patient or to listen to the audio dataset in real-time by muting, fully or partly, either the real-time voice communication or the audio dataset.

### Optional Features:

the system is configured to use the speech microphone to determine unwanted noise or noise that otherwise affects the quality of the audio dataset and to generate a warning if the unwanted noise exceeds a threshold.
where the intermediary device is a laptop or PC, then the internet-connected app treats the patient speech and the audio dataset generated by the medical device in a way that satisfies the standard browser security model of allowing for multiple audio sources to be used at any given time
where the intermediary device is a smartphone or smartwatch, then the internet-connected app processes both the patient speech and also the audio dataset generated by the medical device in a way that satisfies the standard smartphone or smartwatch model of allowing for multiple audio sources to be used at any given time only if they are integrated into a single app.
the speech and audio datasets are each delivered over a stereo channel and the web app separates the audio signal into two separate mono feeds and processes each differently.
the clinically relevant audio dataset channel has a gain control to increase the strength of the signal.
the clinically relevant audio datasets to improve the quality of the audio from a clinical or diagnostic perspective.
filters are applied to the speech sounds and also the clinically relevant sounds, after these sounds have been recorded, maintaining a raw audio file or files.
the healthcare professional and/or patient each have control of muting the speech channel and the clinically relevant sound channel separately if they want to only hear one or the other channel.
the speech microphone is used to capture audio that is used to reduce or remove sounds that are not relevant to the clinically relevant sound channel and hence the audio dataset.
the speech microphone output is used to determine if the room is too noisy for a patient reading and/or if a patient is speaking when the exam is being recorded to enable a message to be shown or given to the patient to be silent and/or that there is too much noise to perform the examination.

### The Medical Device is a Digital Stethoscope

the medical device is a digital stethoscope and the clinically relevant sound are auscultation sounds.
The audio dataset channel, e.g. auscultation sound channel, has a gain control so a strong enough signal will be captured for the body recording.
The digital stethoscope connects to the intermediary device using a USB port.
the digital stethoscope connects to the intermediary device using short-range wireless.
the digital stethoscope includes a single visual output and a single button.
the digital stethoscope is waterproof.
digital stethoscope comprises a first audio sensor that is configured to measure or sense body sounds and a second audio sensor that is configured to measure or sense sounds from the patient or the environment around the patient.
the remote web server is configured to generate a unique web-link that is associated with a specific patient dataset; and in which the unique web-link enables a healthcare professional to review the specific patient dataset by selecting the web-link from within a web browser or from within any dedicated telemedicine application that opens web-links.

Another aspect is a medical device that includes (i) a speech microphone configured to detect and/or record patient speech and (ii) a second microphone configured to detect and/or record clinically relevant sounds and generate an audio dataset from those sounds;
in which the speech microphone uses one channel of a stereo channel pair, and the second microphone uses the other channel, and each channel is processed substantially in parallel or simultaneously.

### Optional Features

the medical device is a digital stethoscope and the clinically relevant sounds are auscultation sounds.
each channel is processed substantially in parallel or simultaneously to enable noise reduction/cancellation techniques.
the noise reduction/cancellation techniques involve measuring the timing/phasing of noise detected by the speech microphone compared with the same noise detected by the auscultation microphone.
each channel is processed substantially in parallel or simultaneously to enable compensating for different timing in receiving auscultation sounds in patients with different body masses.
the system is configured to use the speech microphone to determine unwanted noise or noise that otherwise affects the quality of the audio dataset and to generate a warning if the unwanted noise exceeds a threshold.
the clinically relevant audio datasets is processed to improve the quality of the audio from a clinical or diagnostic perspective.
the clinically relevant audio dataset channel has a gain control to increase the strength of the signal.
filters are applied to the speech sounds and also the clinically relevant sounds, after these sounds have been recorded, maintaining a raw audio file or files.
a healthcare professional and/or patient each have control of muting the speech channel and the clinically relevant sound channel separately if they want to only hear one or the other channel.
the speech microphone is used to capture audio that is used to reduce or remove sounds that are not relevant to the clinically relevant sound channel and hence the audio dataset.
the speech microphone output is used to determine if the room is too noisy for a patient reading and/or if a patient is speaking when the exam is being recorded to enable a message to be shown or given to the patient to be silent and/or that there is too much noise to perform the examination.
each channel is processed substantially in parallel or simultaneously to enable noise reduction/cancellation techniques at the medical device.
the medical device is configured to upload or send patient datasets to a remote web server, directly from an internet-connected app running either on the device or on an intermediary device
each channel is processed substantially in parallel or simultaneously to enable noise reduction/cancellation techniques at the intermediary device
where the intermediary device is a laptop or PC, then the patient speech and the audio dataset generated by the medical device are treated in a way that satisfies the standard browser security model of allowing for multiple audio sources to be used at any given time
where the intermediary device is a smartphone or smartwatch, then the patient speech and also the audio dataset generated by the medical device are treated in a way that satisfies the standard smartphone or smartwatch model of allowing for multiple audio sources to be used at any given time only if they are integrated into a single app.
the medical device is a single, unitary device and the speech microphone and the second microphone are integrated into that single, unitary device.
the medical device comprises two physically separate or separable units, and the speech microphone and the second microphone are integrated into different separate or separable units.

### Feature 3: Healthcare Applet: a High Level Healthcare Programming Environment

The Medaica system is able to generate advice or instructions on when to perform specific healthcare management protocols, such as when specific bodily sounds or functions should be measured. In previous examples, we described how a low-cost stethoscope could be connected to a patient's smartphone which could in turn send audio etc. recordings to a remote server. In those earlier scenarios, the patient is taken to be manually placing the stethoscope at positions on his or her body that the patient hopes are correct. In the Medaica system, the patient can be guided, by an application running on the smartphone, to position the device at different positions and to then create a recording from each of those positions. For example, the application could provide voice instructions to the patient, such as ‘first, place your stethoscope over the heart and press record’. The application could display a graphic indicating on an image of a body where to place the stethoscope. Once that recording has been made, the application could provide another spoken instruction such as ‘Now, move the stethoscope down 5 cm”; again a graphic could be shown to guide the patient. The guidance could be timed, so that, for example, at two or three pre-set times each day, the patient would be guided through the steps needed to use the stethoscope in the ways dictated by a protocol set by the patient's doctor.

We can generalize to:

A telemedicine system comprising multiple medical devices that are each configured to generate patient datasets, and a remote web server; in which:
a medical device is configured to upload or send patient datasets to the remote web server, directly from an internet-connected app running either on the device or on an intermediary device;
and in which the remote web server hosts or enables access to an applet that, when run on the internet-connected app, provides instructions or guides to the patient to perform specific healthcare management protocols.

### Optional Features:

the applet guides the patient to take specific tests with a specific frequency
the applet sends reminders to the patient as well as updates to the patient's healthcare provider(s) and/or insurer or other parties with appropriate permissions.
the applet guides the patient to use a digital stethoscope in a specific position, for a specific duration and frequency.
the applet sends reminders to the patient as well as updates to the patient's healthcare provider(s) and/or insurer or other parties with appropriate permissions.
the applet guides the patient to use a digital stethoscope in a specific position.
the applet guides the patient to use a digital stethoscope in a specific position, for a specific duration and frequency.
the applet provides instructions or guides with a diagram, animation or video.
the applet sends patient datasets to the healthcare professional
the applet monitors compliance with the instructions or guides it provides to the patient
the applet is a Patient Release Protocol that provides instructions or guides to the patient to perform specific healthcare management protocols relevant to their release from hospital
the applet integrate patient datasets generated in response to the applet into their healthcare records of the relevant patient.
the applet provides a protocol for a clinical trials.
the remote web server is configured to generate a unique web-link that is associated with a specific patient dataset; and in which the unique web-link enables a healthcare professional to review the specific patient dataset by selecting the web-link from within a web browser or from within any dedicated telemedicine application that opens web-links.

### Feature 4: Virtual Healthcare Exam Systems and Methods

The Medaica system enables healthcare professionals to directly conduct remote examination using a virtual examination room hosted on a remote web server. For example, extending the use cases described above, the doctor can open a virtual examination video room, invite the patient to join, and conduct a virtual examination by asking the patient to move the stethoscope to specific areas and select ‘record’; the audio recording can be streamed to the remote server, and added to the resources available to the doctor in the virtual examination room so that the doctor can listen to the recording in real-time. The doctor can ask the patient to repeat the recording, or guide the patient to move the stethoscope to a new position, and create a new recording, which can be listened to in real-time. The doctor can edit the recording to eliminate clinically irrelevant sections and can then share a web-link that includes that edited audio file, for example with experts for a second opinion.

We can generalize to:

A telemedicine system comprising multiple medical devices that are each configured to generate patient datasets, and a remote web server connected to each medical device; in which:
a medical device is configured to upload or send patient datasets to the remote web server, directly from an internet-connected app running either on the device or on an intermediary device;
in which the system is configured to enable a healthcare professional and a patient to communicate via a virtual examination room, and the system is further configured to display a user interface that includes a virtual or graphical body image or body outline and one or more target positions at which a medical device is to be positioned by the patient;
and the system is further configured to enable a dynamic interaction between the patient or the healthcare professional and the user interface, to enable the patient to correctly position the medical device at the target position or positions.

### Optional Features

the system is configured to overlay or integrate a real-time image of the patient with the virtual or graphical body image or body outline to enable a dynamic interaction in which the patient matches or overlaps the two images to enable the patient to position the medical device at the target position or positions.
the system is configured to enable a dynamic interaction in which the healthcare professional alters the location of the target position or positions.
the patient enters that virtual examination room by entering a code, such as a code provided by the healthcare professional and once both healthcare professional and patient are in the same virtual examination room, the healthcare professional and the patient can communicate by voice and/or video.
the system is configured to enable the code to be provided by the healthcare professional to the patient.
healthcare professional and the patient are communicating via the virtual examination room, the healthcare professional can guide the patient into using the medical device in specific ways defined by the examination protocol and the system is further configured to provide feedback if the patient is operating the medical device in compliance with that protocol.
once both healthcare professional and patient are in the same virtual examination room, the patient can use their medical device to create datasets which are uploaded to the remote web server and made available automatically and substantially immediately to the healthcare professional to review and/or record.
the user interface is configured to show a body map or body image of a part of a patient's body with an icon or other mark representing the medical device, in which the icon or mark is movable by a participant in a telemedicine session.
the system is configured to enable the healthcare professional to move the icon or mark on the body map or body image and to display to the patient the moving icon or mark to enable the patient to place his/her medical device to overlay the icon or mark on the body map or body image.
the internet-connected app displays an augmented reality view to guide the patient to find a specific position to place the medical device.
the medical device automatically generates a patient dataset when the medical device is positioned at or near the specific position.
an augmented reality view is provided that includes an outline of the patient based on sensor data, and the augmented reality view is displayed to both the patient and healthcare professional at the same time.
the internet-connected app displays an outline of a torso or other part of the body in a video feed and indicates a specific position on the torso or other body part at which the patient is to place the medical device.
the system is configured to provide a patient self-examination mode, in which different target positions, at which the medical device is to be placed, are shown or indicated to the patient on the internet-connected app; and the system is configured to create, manually or automatically, a patient dataset or recording at each specific position.
different target positions are sequentially displayed after each patient dataset or recording at a target position has been completed.
some or all of the target positions are medically standard positions or are specifically chosen by the healthcare professional.
the medical device is a stethoscope and the target positions are specific, standard auscultation positions, or, if the patient is receiving guidance from the healthcare professional, the desired auscultation positions can be moved by the healthcare professional in real time.
data defining the target positions is recorded as part of the related patient dataset.
the patient dataset is an audio or video file or stream.
the patient dataset is an auscultation audio or video file or stream.
the patient dataset is data relating to the heart, lung or any other organ.
the remote web server is configured to generate a unique web-link that is associated with a specific patient dataset.
the unique web-link is configured to enable a healthcare professional to review the specific patient dataset by selecting the web-link from within a web browser or from within any dedicated telemedicine application that opens web-links and to initiate a virtual examination of the patient by opening a link to a virtual examination room hosted on the remote web server.

Other generally applicable optional features include:

### Doctor Guided Device Icon (Simple Non-AR Implementation)

An app (including a web) view shows a “a body map” being a patient's upper body (e.g. an outline) with an icon/mark representing the device (e.g. a stethoscope) that is movable by a participant (usually the healthcare provider) in the telemedicine session.
The doctor can move the icon/mark on the body map, such that the patient sees the mark moving and the patient can then place his/her device (in the real world) to overlay the mark on the body map to position the device accurately and correctly.

### Augmented Reality (AR)

the patient's web-app displays an augmented reality view to guide the patient to find a specific position to place the digital medical device.
an augmented reality view includes an outline of the end-user based on sensor data, such as camera or LIDAR data and the augmented reality view is displayed to the both the patient and healthcare professional at the same time.
the digital medical device automatically generates a patient dataset when the digital medical device is positioned at or near the specific location.

### Assisted Exam Interface

Similar to the AR embodiment described above, a patient's web-app displays an image or outline of a torso in its video feed. The patient positions him/herself into or within the torso image or outline, and is then guided to place the digital medical device at specific position(s) (such as auscultation positions where the device is a stethoscope).
In a self-exam mode, the (e.g. auscultation) positions can be sequentially displayed to the patient after each has been recorded. Alternatively, if a specific sequence has been requested by the healthcare professional, that sequence can be displayed.
If the patient is receiving guidance from the healthcare professional, the positions can be altered or moved by the healthcare professional in real time.
Each position can be recorded alongside the audio file as tagged references, to further assist in diagnosis and records.

### Patient Dataset

the patient dataset is a file or stream
the patient dataset is an audio or video file or stream
the patient dataset is an auscultation audio or video file or stream
the patient dataset is data relating to the heart, lung or any other organ.

### Security

web link is associated with one or more use restrictions.
use restrictions includes: a time period for accessing the shareable link, predefined number of times the web link is accessible, authorized third party, compression format, sharing rights, downloading rights, payments.
use restrictions includes enabling the decryption of the patient datasets.
use restrictions includes enabling diagnostic analysis
each patient dataset is encrypted before being saved at the remote web server location.
each patient dataset is associated with a secure unique ID.
a secure unique ID is linked to an end-user and a unique device ID.
a secure unique ID is only identifiable by the healthcare professional.
web-link only provides access to encrypted patient dataset.
encrypted patient datasets can only be decrypted by authorized third party.

### Blockchain

the method uses a blockchain server to store patient datasets.
only authorized third party can have access to the blockchain server
blockchain server stores an audit trail of all events associated with each patient dataset.

### Note

It is to be understood that the above-referenced arrangements are only illustrative of the application for the principles of the present invention. Numerous modifications and alternative arrangements can be devised without departing from the spirit and scope of the present invention. While the present invention has been shown in the drawings and fully described above with particularity and detail in connection with what is presently deemed to be the most practical and preferred example(s) of the invention, it will be apparent to those of ordinary skill in the art that numerous modifications can be made without departing from the principles and concepts of the invention as set forth herein.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. A telemedicine system comprising: multiple medical devices that are each configured to generate patient datasets, and a remote web server connected to each medical device; in which:
a medical device is configured to upload or send patient datasets to the remote web server, directly from an internet-connected app running either on the device or on an intermediary device;
and in which the medical device includes (i) a speech microphone configured to detect and/or record patient speech and (ii) a second microphone in the medical device configured to detect and/or record clinically relevant sounds and generate an audio dataset from those sounds;
and in which the internet-connected app is configured to treat that patient speech separately from the audio dataset and is hence configured to enable real-time voice communication from the patient to a healthcare professional at the same time as the audio dataset is being shared with the healthcare professional via the remote web server;
and the system is configured to enable the healthcare professional to select whether to listen to real-time voice communication from the patient or to listen to the audio dataset in real-time by muting, fully or partly, either the real-time voice communication or the audio dataset.

32. The telemedicine system of claim 31, in which the system is configured to use the speech microphone to determine unwanted noise or noise that otherwise affects the quality of the audio dataset and to generate a warning if the unwanted noise exceeds a threshold.

33. The telemedicine system of claim 31, where the intermediary device is a laptop or PC, then the internet-connected app treats the patient speech and the audio dataset generated by the medical device in a way that satisfies the standard browser security model of allowing for multiple audio sources to be used at any given time.

34. The telemedicine system of claim 31, where the intermediary device is a smartphone or smartwatch, then the internet-connected app processes both the patient speech and also the audio dataset generated by the medical device in a way that satisfies the standard smartphone or smartwatch model of allowing for multiple audio sources to be used at any given time only if they are integrated into a single app.

35. The telemedicine system of claim 31, in which the speech and audio datasets are each delivered over a stereo channel and the web app separates the audio signal into two separate mono feeds and processes each differently.

36. The telemedicine system of claim 31, in which the clinically relevant audio dataset channel has a gain control to increase the strength of the signal.

37. The telemedicine system of claim 31, in which the clinically relevant audio datasets are processed to improve the quality of the audio from a clinical or diagnostic perspective.

38. The telemedicine system of claim 31, in which filters are applied to the speech sounds and also the clinically relevant sounds, after these sounds have been recorded, maintaining a raw audio file or files.

39. The telemedicine system of claim 31, in which the healthcare professional and/or patient each have control of muting the speech channel and the clinically relevant sound channel separately if they want to only hear one or the other channel.

40. The telemedicine system of claim 31, in which the speech microphone is used to capture audio that is used to reduce or remove sounds that are not relevant to the clinically relevant sound channel and hence the audio dataset.

41. The telemedicine system of claim 31, in which the speech microphone output is used to determine if the room is too noisy for a patient reading and/or if a patient is speaking when the exam is being recorded to enable a message to be shown or given to the patient to be silent and/or that there is too much noise to perform the examination.

42. The telemedicine system of claim 31, in which the medical device is a digital stethoscope and the clinically relevant sound are auscultation sounds.

43. The telemedicine system of claim 31, in which the audio dataset channel has a gain control so a strong enough signal will be captured for the body recording.

44. The telemedicine system of claim 31, in which the digital stethoscope comprises a first audio sensor that is configured to measure or sense body sounds and a second audio sensor that is configured to measure or sense sounds from the patient or the environment around the patient.

45. The telemedicine system of claim 31, in which the remote web server is configured to generate a unique web-link that is associated with a specific patient dataset; and in which the unique web-link enables the healthcare professional to review the specific patient dataset by selecting the web-link from within a web browser or from within any dedicated telemedicine application that opens web-links.

46. A medical device that includes (i) a speech microphone configured to detect and/or record patient speech and (ii) a second microphone configured to detect and/or record clinically relevant sounds and generate an audio dataset from those sounds; in which the speech microphone uses one channel of a stereo channel pair, and the second microphone uses the other channel, and each channel is processed substantially in parallel or simultaneously.

47. The medical device of claim 46, in which each channel is processed substantially in parallel or simultaneously to enable noise reduction and/or cancellation techniques.

48. The medical device of claim 47, in which the noise reduction and/or cancellation techniques involve measuring the timing and/or phasing of noise detected by the speech microphone compared with the same noise detected by the auscultation microphone.

49. The medical device of claim 46, in which each channel is processed substantially in parallel or simultaneously to enable compensating for different timing in receiving auscultation sounds in patients with different body masses.

50. The medical device of claim 46, in which each channel is processed substantially in parallel or simultaneously to enable noise reduction/cancellation techniques at the medical device.

51. The medical device of claim 46, in which each channel is processed substantially in parallel or simultaneously to enable noise reduction/cancellation techniques at the intermediary device.

52. The medical device of claim 46, in which the medical device is a single, unitary device and the speech microphone and the second microphone are integrated into that single, unitary device.

53. The medical device of claim 46, in which the medical device comprises two physically separate or separable units, and the speech microphone and the second microphone are integrated into different separate or separable units.
